(19) United States
(12) Patent Application Publication
(10) Pub. No.: US 2024/0002395 A1
(43) Pub. Date: Jan. 4, 2024

(54) COMPOUNDS AND THEIR USE FOR TREATING NEUROPATHIC PAIN

(71) Applicant: The Johns Hopkins University, Baltimore, MD (US)

(72) Inventors: Takashi Tsukamoto, Baltimor, MD (US); Barbara Slusher, Baltimore, MD (US); Nlyada Hin, Baltimore, MD (US); Camilo Rojas, Baltimore, MD (US); Xinzhong Dong, Baltimore, MD (US); Yun Guan, Baltimore, MD (US); Ilyas Abdul-Quddoos Berhane, Baltimore, MD (US)

(21) Appl. No.: 18/255,767

(22) PCT Filed: Nov. 30, 2021
(86) PCT No.: PCT/US2021/061172

§ 371 Date: Jun. 2, 2023

Related U.S. Application Data
(60) Provisional application No. 63/121,283, filed on Dec. 4, 2020.

(51) Int. Cl. C07D 495/04; C07D 491/048; A61K 45/06; A61P 25/00

(52) U.S. Cl. CPC C07D 495/04; C07D 491/048; A61K 45/06; A61P 25/00

(57) ABSTRACT

Positive allosteric modulators (PAMs) of Mas-related G protein-coupled receptor X1 (MRGPRX1) and their use for treating neuropathic pain is disclosed.

### FEDERALLY SPONSORED RESEARCH OR DEVELOPMENT

This invention was made with government support under grants NS 101954 and NS 115718 awarded by the National Institutes of Health. The government has certain rights in the invention.

### BACKGROUND

Chronic pain remains one of the most prevalent yet undertreated health problems. Doth et al., Pain (2010). In the United States alone, chronic pain affects over 116 million adults and the associated costs (treatment and lost productivity) exceed $500 billion per year, more than those for cancer, heart disease, and diabetes combined. Meghani et al., Pain Medicine (2012). The best available pharmacologic therapies for chronic neuropathic pain leave much to be desired, vis-à-vis both effectiveness and tolerability. Dworkin et al., Mayo Clinic Proceedings (2010). Additionally, the opioid epidemic that currently ravages the U.S. has highlighted this unmet need, spurring the search for safer and more effective analgesics. Kolodny et al., Annu Rev Public Health (2015).

### SUMMARY

In some aspects, the presently disclosed subject matter provides a compound of formula (I):

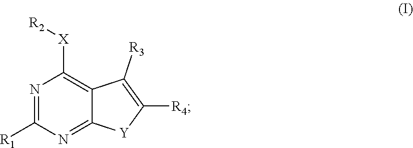

wherein:
X and Y are each independently O or S;
R₁ is selected from the group consisting of H, C₁-C₄ alkyl, C₃-C₇ substituted or unsubstituted cycloalkyl or cycloheteroalkyl, substituted or unsubstituted aryl, and substituted or unsubstituted heteroaryl;
R₂ is selected from the group consisting of C₁-C₄ alkyl, C₃-C₇ substituted or unsubstituted cycloalkyl or cycloheteroalkyl, substituted or unsubstituted aryl, substituted or unsubstituted arylalkyl, and substituted or unsubstituted heteroaryl;
R₃ is selected from the group consisting of H, C₁-C₄ alkyl, C₃-C₇ substituted or unsubstituted cycloalkyl or cycloheteroalkyl, substituted or unsubstituted aryl, substituted or unsubstituted heteroaryl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloalkyl or cycloheteroalkyl, substituted or unsubstituted aryl, substituted or unsubstituted arylalkyl, substituted or unsubstituted heteroaryl, and —OR₈, wherein R₈ is C₁-C₄ alkyl;
R₄ is selected from the group consisting of straightchain or branched substituted or unsubstituted C₁-C₄ alkyl, C₃-C₇ substituted or unsubstituted cycloalkyl or cycloheteroalkyl, substituted or unsubstituted aryl, and substituted or unsubstituted heteroaryl;
provided that R₂ and R₃ or R₃ and R₄, or any substituent groups thereof, do not together form a cyclic ring;
under the further provisos:
(a) when X is O and Y is S:
   (i) R₄ is not a methyl or ethyl group;
   (ii) when R₃ is H and R₄ is a t-butyl group, R₂ cannot be (3-substituted isozazol-5-yl)methyl;
   (iii) when R₄ is an isopropyl group, R₂ cannot be a substituted cyclohexyl group; and
   (iv) when R₄ is a butyl group or a 2-oxopropyl group, R₃ cannot be H;
(b) when X and Y are both S:
   (i) when R₄ is an isopropyl group, R₂ cannot be carboxymethyl or 2-methoxy-2-oxoethyl;
   (ii) when R₄ is a cyclopropyl group, R₂ cannot be carboxymethyl or 2-methoxy-2-oxoethyl;
   (iii) when R₄ is a propyl group, R₃ cannot be chloride; and
   (iv) when R₄ is a 3-ethoxy-3-oxopropyl group, R₃ cannot be H;
(c) when X and Y are both O:
   (i) when R₄ cannot be a methyl group; and
   (ii) when R₄ is a cyclopentyl group, R₃ cannot be 4-methoxyphenyl;
   and
(d) when X is S and Y is O:
   (i) when R₃ is H, R₂ cannot be methyl; and
   (ii) when R₃ is methyl, R₂ cannot be carboxymethyl, 2-methoxy-2-oxoethyl, 4-fluorophenyl, or 2-(3-methyl-4-oxoimidazolidin-1-yl)-2-oxoethyl; and
pharmaceutically acceptable salts thereof.

In some aspects, R₂ is selected from the group consisting of benzyl, phenyl, pyridyl, pyridazinyl, pyrimidinyl, pyrazinyl, pyrrolidinyl, piperidinyl, piperazinyl, and morpholinyl, each of which can be substituted or unsubstituted and wherein the piperazinyl is optionally substituted in the 4-nitrogen position with C₁-C₄ alkyl or acyl.

In particular aspects, the benzyl, phenyl, pyridyl, pyridazinyl, pyrimidinyl, pyrazinyl, pyrrolidinyl, piperidinyl, piperazinyl, and morpholinyl are substituted with one or more of halogen, —CF₃, and —OCF₃.

In some aspects, R₃ is selected from the group consisting of phenyl, pyridyl, pyridazinyl, pyrimidinyl, pyrazinyl, oxazolyl, thiazolyl, pyrrolidinyl, piperidinyl, piperazinyl, and morpholinyl, each of which can be substituted or unsubstituted and wherein the piperazinyl is optionally substituted in the 4-nitrogen position with C₁-C₄ alkyl or acyl.

In particular aspects, the phenyl, pyridyl, pyridazinyl, pyrimidinyl, pyrazinyl, oxazolyl, thiazolyl, pyrrolidinyl, piperidinyl, piperazinyl, and morpholinyl are substituted with one or more of C₁-C₄, halogen, —CF₃, and —OCF₃.

In some aspects, R₃ is selected from the group consisting of C₃-C₇ substituted or unsubstituted cycloheteroalkyl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloheteroalkyl, and —OR₈, wherein R₈ is C₁-C₄ alkyl.

In particular aspects of the compound of formula (I), X is O, R₄ is t-butyl, and the compound of formula (I) is:

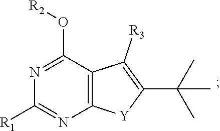

wherein:
Y is O or S;
R₁ is selected from the group consisting of H, C₁-C₄ alkyl, amino, and substituted or unsubstituted heteroaryl;
R₂ is selected from the group consisting of C₁-C₄ alkyl, C₃-C₇ substituted or unsubstituted cycloalkyl, and substituted or unsubstituted aryl;
R₃ is selected from the group consisting of H, C₁-C₄ alkyl, C₃-C₇ substituted or unsubstituted cycloalkyl or cycloheteroalkyl, substituted or unsubstituted aryl, substituted or unsubstituted heteroaryl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloalkyl or cycloheteroalkyl, substituted or unsubstituted aryl, substituted or unsubstituted arylalkyl, substituted or unsubstituted heteroaryl, and —OR₈, wherein R₈ is C₁-C₄ alkyl;
under the proviso that R₁ and R₃ cannot both be H if R₂ is methyl or 3-substituted (isoxazole-5-yl)methyl; and
pharmaceutically acceptable salt thereof.

In more particular aspects, the compound of formula (I) is:

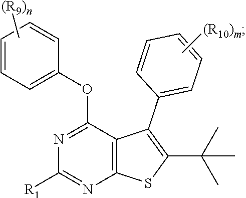

wherein:
m and n are each independently an integer selected from the group consisting of 0, 1, 2, 3, 4, and 5;
R₁ is selected from the group consisting of H, C₁-C₄ alkyl, amino, and substituted or unsubstituted heteroaryl;
each R₉ is independently selected from the group consisting of H, C₁-C₄ alkyl, halogen, —CF₃, —OCF₃, C₁-C₄ alkoxyl, substituted or unsubstituted aryl, substituted or unsubstituted cycloheteroalkyl, substituted or unsubstituted aryloxyl, and —C(═O)—R₁₁, wherein R₁₁ is C₁-C₄ alkyl; and
each R₁₀ is independently selected from the group consisting of halogen, C₁-C₄ alkyl, —CF₃, —OCF₃, C₁-C₄ alkoxyl, and one or more electron withdrawing groups selected from the group consisting of trifluoromethylsulfonyl, nitro, sulfonic acid, —SO₂R₁₂, cyano, formyl, —C(═O)—R₁₃, carboxyl, —CO₂R₁₄, aminocarbonyl, and nitroso, wherein R₁₂, R₁₃ and R₁₄ are each independently C₁-C₄ alkyl.

In some aspects, R₃ is phenyl substituted with one or more halogens. In particular aspects, R₃ is selected from the group consisting of 3-chlorophenyl, 4-chlorophenyl, 3-fluorophenyl, 4-fluorophenyl, 3,4-dichlorophenyl, and 3,4-difluorophenyl.

In some aspects, R₂ is selected from the group consisting of phenyl, pyridinyl, and pyridazinyl, wherein the phenyl, pyridinyl, and pyridazinyl can be unsubstituted or substituted with one or more substituents selected from the group consisting of C₁-C₄ branched or straightchain alkyl, halogen, trifluoromethoxyl, 2,2,2-trifluoroethoxyl, nitro, C₁-C₄-alkoxyl, amino, cyano, substituted or unsubstituted C₃-C₇ cycloalkyl or cycloheteroalkyl, bicycloalkyl, and —C(═O)—R₄, wherein R₄ is C₁-C₄ alkyl.

In particular aspects, R₂ is selected from the group consisting of isopropyl, 2 methylphenyl, phenyl, 2-fluorophenyl, 3-fluorophenyl, 4-fluorophenyl, 2,6-difluorophenyl, 2-fluoro-6-trifluoromethoxyphenyl, 2-trifluoromethoxy-6-fluorophenyl, 2-methoxyphenyl, 2-ethoxyphenyl, 2-trifluoromethoxyphenyl, 2-(2,2,2-trifluoroethoxy)phenyl, 2-phenylethan-1-one, 2-cyanophenyl, 3-trifluoromethoxyphenyl, 4-trifluoromethoxyphenyl, 2-fluoro-6-trifluoromethoxyphenyl, 2-chloro-6-trifluoromethoxyphenyl, 4-nitro-2-trifluoromethoxyphenyl, 4-(4-(piperazin-1-yl)phenoxy)phenyl, 4-(4-(trifluoromethyl)phenoxy)phenyl, 2-fluoropyridin-3-yl, 2-(trifluoromethoxy)pyridin-3-yl, 2-methoxypyridin-3-yl, 2-aminopyridin-3-yl, pyridazin-4-yl, cyclopropyl, cyclobutyl, cyclopentyl, cyclohexyl, cycloheptyl, 1-methylpiperidin-3-yl, 2 methylcyclohexyl, 2-trifluoromethylcyclohexyl, 2-fluorocyclohexyl, 2,2-dimethylcyclohexyl, and 1-trifluoromethylcyclohexyl, bicyclo[2.2.1]heptan-2-yl.

In certain aspects, the compound of formula (I) is:

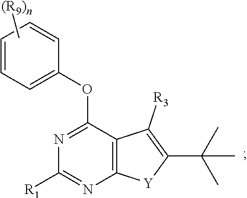

wherein:
n is 2;
Y is O or S;
R₁ is H;
each R₉ is independently F or —OF₃; and
R₃ is selected from the group consisting of C₃-C₇ substituted or unsubstituted cycloheteroalkyl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloheteroalkyl, and —OR₈, wherein R₈ is C₁-C₄ alkyl.

In more particular aspects, the compound is selected from the group consisting of: 6-(tert-butyl)-4-(2-fluorophenoxy)thieno[2,3-d]pyrimidine; 6-(tert-butyl)-4-(2 fluorophenoxy)-5-phenylthieno[2,3-d]pyrimidine; 6-tert-butyl-5-(3,4-dichlorophenyl)-4-(2-(trifluoromethoxy)phenoxy)thieno[2,3-d]pyrimidine; 6-tert-butyl-5-(3,4-dichlorophenyl)-4-(2-fluoro-6-(trifluoromethoxy)phenoxy)thieno[2,3-d]pyrimidine; 6-tert-butyl-4-(2-chloro-6-(trifluoromethoxy)phenoxy)-5-(3,4-dichlorophenyl)thieno[2,3-d]pyrimidine; 6-tert-butyl-5-(3,4-dichlorophenyl)-4-(3-(trifluoromethoxy)phenoxy)thieno[2,3-d]pyrimidine; 6-tert-butyl-5-(3,4-dichlorophenyl)-4-(4-(trifluoromethoxy)phenoxy)thieno[2,3-d]pyrimidine; 6-tert-butyl-5-(3,4-dichlorophenyl)-4-(4-nitro-2-(trifluoromethoxy)phenoxy)thieno[2,3-d]pyrimidine; 6-tert-butyl-5-(3,4-dichlorophenyl)-4-(2-fluoropyridin-3-yloxy)thieno[2,3-d]pyrimidine; 6-tert-butyl-5-(3,4-dichlorophenyl)-4-(2-(trifluoromethoxy)pyridin-3-yloxy)thieno[2,3-d]pyrimidine; 6-tert-butyl-5-(3,4-dichlorophenyl)-4-(2-methoxypyridin-3-yloxy)thieno[2,3-d]pyrimidine; 3-(6-tert-butyl-5-(3,4-dichlorophenyl)thieno[2,3-d]pyrimidin-4-yloxy)pyridin-2-amine; 6-tert-butyl-5-(3,4-dichlorophenyl)-4-(pyridazin-4-yloxy)thieno[2,3-d]pyrimidine; 6-tert-butyl-5-(3,4-dichlorophenyl)-4-phenoxythieno[2,3-d]pyrimidine; 6-tert-butyl-5-(3,4 dichlorophenyl)-4-(2-fluorophenoxy)thieno[2,3-d]pyrimidine; 6-tert-butyl-5-(3,4-dichlorophenyl)-4-(2,6-difluorophenoxy)thieno[2,3-d]pyrimidine; 6-tert-butyl-5-(3,4-dichlorophenyl)-4-(3-fluorophenoxy)thieno[2,3-d]pyrimidine; 6-tert-butyl-5-(3,4-dichlorophenyl)-4-(4-fluorophenoxy)thieno[2,3-d]pyrimidine; 6-tert-butyl-5-(3,4-dichlorophenyl)-4-(2-methoxyphenoxy)thieno[2,3-d]pyrimidine; 6-tert-butyl-5-(3,4 dichlorophenyl)-4-(2-ethoxyphenoxy)thieno[2,3-d]pyrimidine; 6-tert-butyl-5-(3,4-dichlorophenyl)-4-(2-(2,2,2-trifluoroethoxy)phenoxy)thieno[2,3-d]pyrimidine; 6-tert-butyl-5-(3,4-dichlorophenyl)-4-(o-tolyloxy)thieno[2,3-d]pyrimidine; 1-(2-(6-tert-butyl-5-(3,4-dichlorophenyl)thieno[2,3-d]pyrimidin-4-yloxy)phenyl)ethanone; 2-(6-tert-butyl-5-(3,4-dichlorophenyl)thieno[2,3-d]pyrimidin-4-yloxy)benzonitrile; 6-tert-butyl-5-(3,4-dichlorophenyl)-4-isopropoxythieno[2,3-d]pyrimidine; 6-tert-butyl-4-cyclopropoxy-5-(3,4-dichlorophenyl)thieno[2,3-d]pyrimidine; 6-tert-butyl-4-cyclobutoxy-5-(3,4-dichlorophenyl)thieno[2,3-d]pyrimidine; 6-tert-butyl-4-(cyclopentyloxy)-5-(3,4-dichlorophenyl)thieno[2,3-d]pyrimidine; 6-tert-butyl-4-(cyclohexyloxy)-5-(3,4-dichlorophenyl)thieno[2,3-d]pyrimidine; 6-tert-butyl-5-(3,4-diflhorophenyl)-4-(1-methylpiperidin-3-yloxy)thieno[2,3-d]pyrimidine; 6-tert-butyl-5-(3,4-dichlorophenyl)-4-(2-methylcyclohexyloxy)thieno[2,3-d]pyrimidine; 4-(Bicyclo[2.2.1]heptan-2-yloxy)-6-tert-butyl-5-(3,4-dichlorophenyl)thieno[2,3-d]pyrimidine; 6-tert-butyl-4-(cyclohepyloxy)-5-(3,4-dichlorophenyl)thieno[2,3-d]pyrimidine; 6-tert-butyl-5-(3,4-diflhorophenyl)-4-(2-(trifluoromethyl)cyclohexyloxy)thieno[2,3-d]pyrimidine; 6-tert-butyl-5-(3,4-dichlorophenyl)-4-(2-fluorocyclohexyloxy)thieno[2,3-d]pyrimidine; 6-tert-butyl-5-(3,4-dichlorophenyl)-4-(2,2-dimethylcyclohexyloxy)thieno[2,3-d]pyrimidine; 6-tert-butyl-5-(3,4-diflhorophenyl)-4-(1-(trifluoromethyl)cyclohexyloxy)thieno[2,3-d]pyrimidine; 6-tert-butyl-5-(3,4-difluorophenyl)-4-(2-fluorophenoxy)thieno[2,3-d]pyrimidine; 6-tert-butyl-4-(2,6-difluorophenoxy)-5-(3,4-difluorophenyl)thieno[2,3-d]pyrimidine; 6-tert-butyl-5-(3,4-difluorophenyl)-4-(2-fluoro-6-(trifluoromethoxy)phenoxy)thieno[2,3-d]pyrimidine; 6-tert-butyl-5-(3,4-difluorophenyl)-4-(2-(trifluoromethoxy)phenoxy)thieno[2,3-d]pyrimidine; 6-tert-butyl-5-(4-chlorophenyl)-4-(2-(trifluoromethoxy)phenoxy)thieno[2,3-d]pyrimidine; 6-tert-butyl-5-(4-chlorophenyl)-4-(2-fluoro-6-(trifluoromethoxy)phenoxy)thieno[2,3-d]pyrimidine; 6-tert-butyl-5-(4-chlorophenyl)-4-(2,6-difluorophenoxy)thieno[2,3-d]pyrimidine; 6-tert-butyl-5-(3-chlorophenyl)-4-(2-(trifluoromethoxy)phenoxy)thieno[2,3-d]pyrimidine; 6-tert-butyl-5-(3-chlorophenyl)-4-(2-fluoro-6-(trifluoromethoxy)phenoxy)thieno[2,3-d]pyrimidine; 6-tert-butyl-5-(3-chlorophenyl)-4-(2,6-difluorophenoxy)thieno[2,3-d]pyrimidine; 6-tert-butyl-4-(2,6-difluorophenoxy)-5-(4-fluorophenyl)thieno[2,3-d]pyrimidine; 6-tert-butyl-4-(2-fluorophenoxy)-5-(4-fluorophenyl)thieno[2,3-d]pyrimidine 6-tert-butyl-5-(3-fluorophenyl)-4-(2-(trifluoromethoxy)phenoxy)thieno[2,3-d]pyrimidine; 6-tert-butyl-4-(2,6-difluorophenoxy)-5-(3-fluorophenyl)thieno[2,3-d]pyrimidine; 6-tert-butyl-5-(3,4-diflhorophenyl)-4-(2-(trifluoromethoxy)phenylthio)thieno[2,3-d]pyrimidine; ethyl 6-(tert-butyl)-4-(2-fluoro-6-(trifluoromethoxy)phenoxy)thieno[2,3-d]pyrimidine-5-carboxylate; 6-(tert-butyl)-4-(2-fluoro-6-(trifluoromethoxy)phenoxy)-5-(pyrrolidin-1-yl)thieno[2,3-d]pyrimidine; 6-(tert-butyl)-4-(2-fluoro-6-(trifluoromethoxy)phenoxy)-5-(2-methylpyrrolidin-1-yl)thieno[2,3-d]pyrimidine; 6-(tert-butyl)-4-(2-fluoro-6-(trifluoromethoxy)phenoxy)-5-(piperidin-1-yl)thieno[2,3-d]pyrimidine; 4-(6-(tert-butyl)-4-(2,6-difluorophenoxy)thieno[2,3-d]pyrimidin-5-yl)morpholine; 6-(tert-butyl)-N,N-diethyl-4-(2-fluoro-6-(trifluoromethoxy)phenoxy)thieno[2,3-d]pyrimidin-5-amine; and (6-(tert-butyl)-4-(2-fluoro-6-(trifluoromethoxy)phenoxy)furo[2,3-d]pyrimidin-5-yl)(piperidin-1-yl)methanone.

In other aspects of the compound of formula (I), X is S, Y is S, and R₄ is t-butyl and the compound of formula (I) is:

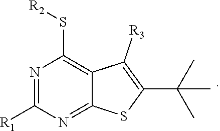

In particular aspects, the compound of formula (I) is:

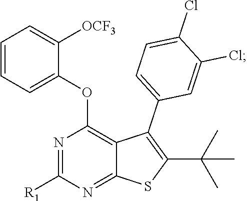

In other aspects of the compound of formula (I), X is O and Y is O and the compound of formula (I) is:

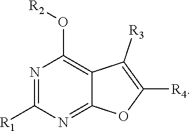

In yet other aspects of the compound of formula (I), X is S and Y is O and the compound of formula (I) is:

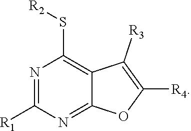

In other aspects, the presently disclosed subject matter provides a method for treating pain in a subject in need of treatment thereof, the method comprising administering to the subject a compound of formula (I), or a pharmaceutically acceptable salt thereof, in a therapeutically effective amount to treat the pain.

In some aspects, the pain comprises neuropathic pain. In some aspects, the neuropathic pain comprises chronic neuropathic pain.

In some aspects, the method further comprises eliminating or attenuating one or more of off-target side effects, opioid-like side effects, and abuse potential. In certain embodiments, the off-target side effect comprises itching.

In some aspects, the compound of formula (I) comprises a positive allosteric modulator of MRGPRX1. In certain aspects, the MRGPRX1 is expressed in one or more DRG neurons.

In some aspects, the presently disclosed method further comprises administering one or more additional therapeutic agents in combination with a compound of formula (I).

In some aspects, the one or more additional therapeutic agents are selected from the group consisting of a therapeutic agent for pain and an anti-inflammatory agent.

In some aspects, the one or more therapeutic agents include a neuropathic disorder agent (e.g., pregabalin), an antidepressant (e.g., duloxetine, amitriptyline), a regional anesthetic (e.g., lidocaine), ketamine, and combinations thereof.

In some aspects, the one or more therapeutic agents comprise an anti-inflammatory agent selected from the group consisting of a steroid (e.g., prednisolone), an antihistamine (e.g., loratadine), and combinations thereof.

In some aspects, the compound of formula (I) is administered systemically.

In some aspects, the compound of formula (I) penetrates the central nervous system.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 shows drug concentration versus time curves in plasma, spinal cord, and brain following oral administration of compound 7a

FIG. 2A shows paw withdrawal latencies (PWLs) to noxious heat stimuli in the ipsilateral (left) and the contralateral (right) hind paws to the side of nerve injury at day 12 after chronic constriction injury (CCI); and

FIG. 2B shows paw withdrawal latencies (PWLs) to noxious heat stimuli in the ipsilateral (left) and the contralateral (right) hind paws to the side of nerve injury at day 26 after chronic constriction injury (CCI).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

A subset of mas-related G protein-coupled receptors (Mrgprs aka Mrgs or SNSRs) is expressed specifically in the small diameter dorsal root ganglia (DRG) sensory neurons. Among these, human homologue MRGPRX1 has been implicated in the modulation of nociception and its agonists will represent a new class of drugs that offer pain relief with minimal side effects owing to its restricted expression in DRG neurons. Dong et al., 2001; Lembo et al., 2002; Zhang et al., 2005. Positive allosteric modulators of MRGPRX1 are particularly promising as they might preferentially activate the central receptors (over the peripheral receptors) due to the preferential generation of endogenous orthosteric MRGPRX1 agonists in the dorsal horn in response to persistent pain, thereby avoiding itch side effects caused by activation of the peripheral receptors. Li et al., 2017. This therapeutic concept was demonstrated by a MRGPRX1 positive allosteric modulator ML382. Wen et al., 2015; Li et al., 2017. ML382, however, showed efficacy in preclinical models of pain by intrathecal injection, limiting its utility in treating chronic pain. Li et al., 2017. Thus, there is a need for orally active positive allosteric modulators of MRGPRX1.

In some embodiments, the presently disclosed subject matter provides a compound of formula (I):

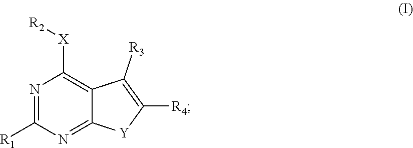

wherein:
X and Y are each independently O or S;
R₁ is selected from the group consisting of H, C₁-C₄ alkyl, C₃-C₇ substituted or unsubstituted cycloalkyl or cycloheteroalkyl, substituted or unsubstituted aryl, and substituted or unsubstituted heteroaryl;
R₂ is selected from the group consisting of C₁-C₄ alkyl, C₃-C₇ substituted or unsubstituted cycloalkyl or cycloheteroalkyl, substituted or unsubstituted aryl, substituted or unsubstituted arylalkyl, and substituted or unsubstituted heteroaryl;
R₃ is selected from the group consisting of H, C₁-C₄ alkyl, C₃-C₇ substituted or unsubstituted cycloalkyl or cycloheteroalkyl, substituted or unsubstituted aryl, substituted or unsubstituted heteroaryl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloalkyl or cycloheteroalkyl, substituted or unsubstituted aryl, substituted or unsubstituted arylalkyl, substituted or unsubstituted heteroaryl, and —OR₈, wherein R₈ is C₁-C₄ alkyl;
R₄ is selected from the group consisting of straightchain or branched substituted or unsubstituted C₁-C₄ alkyl, C₃-C₇ substituted or unsubstituted cycloalkyl or cycloheteroalkyl, substituted or unsubstituted aryl, and substituted or unsubstituted heteroaryl;
provided that R₂ and R₃ or R₃ and R₄, or any substituent groups thereof, do not together form a cyclic ring;
under the further provisos:
(a) when X is O and Y is S:
   (i) R₄ is not a methyl or ethyl group;
   (ii) when R₃ is H and R₄ is a t-butyl group, R₂ cannot be (3-substituted isozazol-5-yl)methyl;
   (iii) when R₄ is an isopropyl group, R₂ cannot be a substituted cyclohexyl group; and
   (iv) when R₄ is a butyl group or a 2-oxopropyl group, R₃ cannot be H;
(b) when X and Y are both S:
   (i) when R₄ is an isopropyl group, R₂ cannot be carboxymethyl or 2-methoxy-2-oxoethyl;
   (ii) when R₄ is a cyclopropyl group, R₂ cannot be carboxymethyl or 2-methoxy-2-oxoethyl;
   (iii) when R₄ is a propyl group, R₃ cannot be chloride; and
   (iv) when R₄ is a 3-ethoxy-3-oxopropyl group, R₃ cannot be H;
(c) when X and Y are both O:
   (i) when R₄ cannot be a methyl group; and
   (ii) when R₄ is a cyclopentyl group, R₃ cannot be 4-methoxyphenyl; and
(d) when X is S and Y is O:
   (i) when R₃ is H, R₂ cannot be methyl; and
   (ii) when R₃ is methyl, R₂ cannot be carboxymethyl, 2-methoxy-2-oxoethyl, 4-fluorophenyl, or 2-(3-methyl-4-oxoimidazolidin-1-yl)-2-oxoethyl; and
pharmaceutically acceptable salts thereof.

In some embodiments, R₂ is selected from the group consisting of benzyl, phenyl, pyridyl, pyridazinyl, pyrimidinyl, pyrazinyl, pyrrolidinyl, piperidinyl, piperazinyl, and morpholinyl, each of which can be substituted or unsubstituted and wherein the piperazinyl is optionally substituted in the 4-nitrogen position with C₁-C₄ alkyl or acyl.

In particular embodiments, the benzyl, phenyl, pyridyl, pyridazinyl, pyrimidinyl, pyrazinyl, pyrrolidinyl, piperidinyl, piperazinyl, and morpholinyl are substituted with one or more of halogen, —CF₃, and —OCF₃.

In some embodiments, R₃ is selected from the group consisting of phenyl, pyridyl, pyridazinyl, pyrimidinyl, pyrazinyl, oxazolyl, thiazolyl, pyrrolidinyl, piperidinyl, piperazinyl, and morpholinyl, each of which can be substituted or unsubstituted and wherein the piperazinyl is optionally substituted in the 4-nitrogen position with C₁-C₄ alkyl or acyl.

In particular embodiments, the phenyl, pyridyl, pyridazinyl, pyrimidinyl, pyrazinyl, oxazolyl, thiazolyl, pyrrolidinyl, piperidinyl, piperazinyl, and morpholinyl are substituted with one or more of C₁-C₄, halogen, —CF₃, and —OCF₃.

In some embodiments, R₃ is selected from the group consisting of C₃-C₇ substituted or unsubstituted cycloheteroalkyl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloheteroalkyl, and —OR₈, wherein R₈ is C₁-C₄ alkyl.

In particular embodiments of the compound of formula (I), X is O, R₄ is t-butyl, and the compound of formula (I) is:

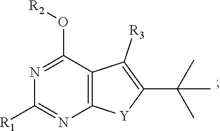

wherein:
Y is O or S;
R₁ is selected from the group consisting of H, C₁-C₄ alkyl, amino, and substituted or unsubstituted heteroaryl;
R₂ is selected from the group consisting of C₁-C₄ alkyl, C₃-C₇ substituted or unsubstituted cycloalkyl, and substituted or unsubstituted aryl;
R₃ is selected from the group consisting of H, C₁-C₄ alkyl, C₃-C₇ substituted or unsubstituted cycloalkyl or cycloheteroalkyl, substituted or unsubstituted aryl, substituted or unsubstituted heteroaryl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloalkyl or cycloheteroalkyl, substituted or unsubstituted aryl, substituted or unsubstituted arylalkyl, substituted or unsubstituted heteroaryl, and —OR₈, wherein R₈ is C₁-C₄ alkyl;
under the proviso that R₁ and R₃ cannot both be H if R₂ is methyl or 3-substituted (isoxazole-5-yl)methyl; and
pharmaceutically acceptable salt thereof.

In more particular embodiments, the compound of formula (I) is:

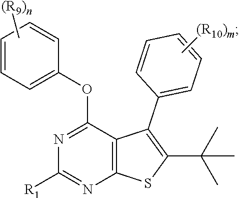

wherein:
m and n are each independently an integer selected from the group consisting of 0, 1, 2, 3, 4, and 5;
R₁ is selected from the group consisting of H, C₁-C₄ alkyl, amino, and substituted or unsubstituted heteroaryl;
each R₉ is independently selected from the group consisting of H, C₁-C₄ alkyl, halogen, —CF₃, —OCF₃, C₁-C₄ alkoxyl, substituted or unsubstituted aryl, substituted or unsubstituted cycloheteroalkyl, substituted or unsubstituted aryloxyl, and —C(═O)—R₁₁, wherein R₁₁ is C₁-C₄ alkyl; and
each R₁₀ is independently selected from the group consisting of halogen, C₁-C₄ alkyl, —CF₃, —OCF₃, C₁-C₄ alkoxyl, and one or more electron withdrawing groups selected from the group consisting of trifluoromethylsulfonyl, nitro, sulfonic acid, SO₂R₁₂, cyano, formyl, —C(═O)—R₁₃, carboxyl, —CO₂R₁₄, aminocarbonyl, and nitroso, wherein R₁₂, R₁₃ and R₁₄ are each independently C₁-C₄ alkyl.

In some embodiments, R₃ is phenyl substituted with one or more halogens. In particular embodiments, R₃ is selected from the group consisting of 3-chlorophenyl, 4-chlorophenyl, 3-fluorophenyl, 4-fluorophenyl, 3,4-dichlorophenyl, and 3,4-difluorophenyl.

In some embodiments, R₂ is selected from the group consisting of phenyl, pyridinyl, and pyridazinyl, wherein the phenyl, pyridinyl, and pyridazinyl can be unsubstituted or substituted with one or more substituents selected from the group consisting of C₁-C₄ branched or straightchain alkyl, halogen, trifluoromethoxyl, 2,2,2-trifluoroethoxyl, nitro, C₁-C₄-alkoxyl, amino, cyano, substituted or unsubstituted C₃-C₇ cycloalkyl or cycloheteroalkyl, bicycloalkyl, and —C(═O)—R₄, wherein R₄ is C₁-C₄ alkyl.

In particular embodiments, R₂ is selected from the group consisting of isopropyl, 2-methylphenyl, phenyl, 2-fluorophenyl, 3-fluorophenyl, 4-fluorophenyl, 2,6-difluorophenyl, 2-fluoro-6-trifluoromethoxyphenyl, 2-trifluoromethoxy-6-fluorophenyl, 2-methoxyphenyl, 2-ethoxyphenyl, 2-trifluoromethoxyphenyl, 2-(2,2,2-trifluoroethoxy)phenyl, 2-phenylethan-1-one, 2-cyanophenyl, 3-trifluoromethoxyphenyl, 4-trifluoromethoxyphenyl, 2-fluoro-6-trifluoromethoxyphenyl, 2-chloro-6-trifluoromethoxyphenyl, 4-nitro-2-trifluoromethoxyphenyl, 4-(4-(piperazin-1-yl)phenoxy)phenyl, 4-(4 (trifluoromethyl)phenoxy)phenyl, 2-fluoropyridin-3-yl, 2-(trifluoromethoxy)pyridin-3-yl, 2-methoxypyridin-3-yl, 2-aminopyridin-3-yl, pyridazin-4-yl, cyclopropyl, cyclobutyl, cyclopentyl, cyclohexyl, cycloheptyl, 1-methylpiperidin-3-yl, 2-methylcyclohexyl, 2-trifluoromethylcyclohexyl, 2-fluorocyclohexyl, 2,2-dimethylcyclohexyl, and 1-trifluoromethylcyclohexyl, bicyclo[2.2.1]heptan-2-yl.

In certain embodiments, the compound of formula (I) is:

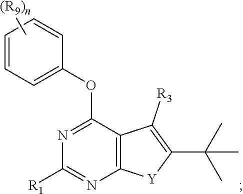

wherein:
n is 2;
Y is O or S;
R₁ is H;
each R₉ is independently F or —OF₃; and
R₃ is selected from the group consisting of C₃-C₇ substituted or unsubstituted cycloheteroalkyl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloheteroalkyl, and —OR₈, wherein R₈ is C₁-C₄ alkyl.

In more particular embodiments, the compound is selected from the group consisting of: 6-(tert-butyl)-4-(2-fluorophenoxy)thieno[2,3-d]pyrimidine; 6-(tert-butyl)-4-(2-fluorophenoxy)-5-phenylthieno[2,3-d]pyrimidine; 6-tert-butyl-5-(3,4-dichlorophenyl)-4-(2-(trifluoromethoxy)phenoxy)thieno[2,3-d]pyrimidine; 6-tert-butyl-5-(3,4-dichlorophenyl)-4-(2-fluoro-6-(trifluoromethoxy)phenoxy)thieno[2,3-d]pyrimidine; 6-tert-butyl-4-(2-chloro-6-(trifluoromethoxy)phenoxy)-5-(3,4-dichlorophenyl)thieno[2,3-d]pyrimidine; 6-tert-butyl-5-(3,4-dichlorophenyl)-4-(3-(trifluoromethoxy)phenoxy)thieno[2,3-d]pyrimidine; 6-tert-butyl-5-(3,4-dichlorophenyl)-4-(4-(trifluoromethoxy)phenoxy)thieno[2,3-d]pyrimidine; 6-tert-butyl-5-(3,4-dichlorophenyl)-4-(4-nitro-2-(trifluoromethoxy)phenoxy)thieno[2,3-d]pyrimidine; 6-tert-butyl-5-(3,4-dichlorophenyl)-4-(2-fluoropyridin-3-yloxy)thieno[2,3-d]pyrimidine; 6-tert-butyl-5-(3,4-dichlorophenyl)-4-(2-(trifluoromethoxy)pyridin-3-yloxy)thieno[2,3-d]pyrimidine; 6-tert-butyl-5-(3,4-dichlorophenyl)-4-(2-methoxypyridin-3-yloxy)thieno[2,3-d]pyrimidine; 3-(6-tert-butyl-5-(3,4-dichlorophenyl)thieno[2,3-d]pyrimidin-4-yloxy)pyridin-2-amine; 6-tert-butyl-5-(3,4-dichlorophenyl)-4-(pyridazin-4-yloxy)thieno[2,3-d]pyrimidine; 6-tert-butyl-5-(3,4-dichlorophenyl)-4-phenoxythieno[2,3-d]pyrimidine; 6-tert-butyl-5-(3,4-dichlorophenyl)-4-(2-fluorophenoxy)thieno[2,3-d]pyrimidine; 6-tert-butyl-5-(3,4-dichlorophenyl)-4-(2,6-difluorophenoxy)thieno[2,3-d]pyrimidine; 6-tert-butyl-5-(3,4 dichlorophenyl)-4-(3-fluorophenoxy)thieno[2,3-d]pyrimidine; 6-tert-butyl-5-(3,4-dichlorophenyl)-4-(4-fluorophenoxy)thieno[2,3-d]pyrimidine; 6-tert-butyl-5-(3,4-dichlorophenyl)-4-(2-methoxyphenoxy)thieno[2,3-d]pyrimidine; 6-tert-butyl-5-(3,4-dichlorophenyl)-4-(2-ethoxyphenoxy)thieno[2,3-d]pyrimidine; 6-tert-butyl-5-(3,4-dichlorophenyl)-4-(2-(2,2,2-trifluoroethoxy)phenoxy)thieno[2,3-d]pyrimidine; 6-tert-butyl-5-(3,4-dichlorophenyl)-4-(o-tolyloxy)thieno[2,3-d]pyrimidine; 1-(2-(6-tert-butyl-5-(3,4-dichlorophenyl)thieno[2,3-d]pyrimidin-4-yloxy)phenyl)ethanone; 2-(6-tert-butyl-5-(3,4-dichlorophenyl)thieno[2,3-d]pyrimidin-4-yloxy)benzonitrile; 6-tert-butyl-5-(3,4-dichlorophenyl)-4-isopropoxythieno[2,3-d]pyrimidine; 6-tert-butyl-4-cyclopropoxy-5-(3,4-dichlorophenyl)thieno[2,3-d]pyrimidine; 6-tert-butyl-4 cyclobutoxy-5-(3,4-dichlorophenyl)thieno[2,3-d]pyrimidine; 6-tert-butyl-4-(cyclopentyloxy)-5-(3,4-dichlorophenyl)thieno[2,3-d]pyrimidine; 6-tert-butyl-4-(cyclohexyloxy)-5-(3,4-dichlorophenyl)thieno[2,3-d]pyrimidine; 6-tert-butyl-5-(3,4-dichlorophenyl)-4-(1-methylpiperidin-3-yloxy)thieno[2,3-d]pyrimidine; 6-tert-butyl-5-(3,4-dichlorophenyl)-4-(2-methylcyclohexyloxy)thieno[2,3-d]pyrimidine; 4 (Bicyclo[2.2.1]heptan-2-yloxy)-6-tert-butyl-5-(3,4-dichlorophenyl)thieno[2,3-d]pyrimidine; 6-tert-butyl-4-(cyclohepyloxy)-5-(3,4-dichlorophenyl)thieno[2,3-d]pyrimidine; 6-tert-butyl-5-(3,4-dichlorophenyl)-4-(2-(trifluoromethyl)cyclohexyloxy)thieno[2,3-d]pyrimidine; 6-tert-butyl-5-(3,4-dichlorophenyl)-4-(2-fluorocyclohexyloxy)thieno[2,3-d]pyrimidine; 6-tert-butyl-5 (3,4-dichlorophenyl)-4-(2,2-dimethylcyclohexyloxy)thieno[2,3-d]pyrimidine; 6-tert-butyl-5-(3,4-dichlorophenyl)-4-(1-(trifluoromethyl)cyclohexyloxy)thieno[2,3-d]pyrimidine; 6-tert-butyl-5-(3,4-difluorophenyl)-4-(2-fluorophenoxy)thieno[2,3-d]pyrimidine; 6-tert-butyl-4-(2,6-difluorophenoxy)-5-(3,4-difluorophenyl)thieno[2,3-d]pyrimidine; 6-tert-butyl-5-(3,4-difluorophenyl)-4-(2-fluoro-6-(trifluoromethoxy)phenoxy)thieno[2,3-d]pyrimidine; 6-tert-butyl-5-(3,4-difluorophenyl)-4-(2-(trifluoromethoxy)phenoxy)thieno[2,3-d]pyrimidine; 6-tert-butyl-5-(4-chlorophenyl)-4-(2-(trifluoromethoxy)phenoxy)thieno[2,3-d]pyrimidine; 6-tert-butyl-5-(4-chlorophenyl)-4-(2-fluoro-6-(trifluoromethoxy)phenoxy)thieno[2,3-d]pyrimidine; 6-tert-butyl-5-(4-chlorophenyl)-4-(2,6-difluorophenoxy)thieno[2,3-d]pyrimidine; 6-tert-butyl-5-(3-chlorophenyl)-4-(2-(trifluoromethoxy)phenoxy)thieno[2,3-d]pyrimidine; 6-tert-butyl-5-(3-chlorophenyl)-4-(2-fluoro-6-(trifluoromethoxy)phenoxy)thieno[2,3-d]pyrimidine; 6-tert-butyl-5-(3-chlorophenyl)-4-(2,6-difluorophenoxy)thieno[2,3-d]pyrimidine; 6-tert-butyl-4-(2,6-difluorophenoxy)-5-(4-fluorophenyl)thieno[2,3-d]pyrimidine; 6-tert-butyl-4-(2-fluorophenoxy)-5-(4-fluorophenyl)thieno[2,3-d]pyrimidine 6-tert-butyl-5-(3-fluorophenyl)-4-(2-(trifluoromethoxy)phenoxy)thieno[2,3-d]pyrimidine; 6-tert-butyl-4-(2,6-difluorophenoxy)-5-(3-fluorophenyl)thieno[2,3-d]pyrimidine; 6-tert-butyl-5-(3,4-dichlorophenyl)-4-(2-(trifluoromethoxy)phenylthio)thieno[2,3-d]pyrimidine; ethyl6-(tert-butyl)-4-(2-fluoro-6-(trifluoromethoxy)phenoxy)thieno[2,3-d]pyrimidine-5-carboxylate; 6-(tert-butyl)-4-(2-fluoro-6-(trifluoromethoxy)phenoxy)-5-(pyrrolidin-1-yl)thieno[2,3-d]pyrimidine; 6-(tert-butyl)-4-(2-fluoro-6-(trifluoromethoxy)phenoxy)-5-(2-methylpyrrolidin-1-yl)thieno[2,3-d]pyrimidine; 6-(tert-butyl)-4-(2-fluoro-6-(trifluoromethoxy)phenoxy)-5-(piperidin-1-yl)thieno[2,3-d]pyrimidine; 4-(6-(tert-butyl)-4-(2,6-difluorophenoxy)thieno[2,3-d]pyrimidin-5-yl)morpholine; 6-(tert-butyl)-N,N-diethyl-4-(2-fluoro-6-(trifluoromethoxy)phenoxy)thieno[2,3-d]pyrimidin-5-amine; and (6-(tert-butyl)-4-(2-fluoro-6-(trifluoromethoxy)phenoxy)furo[2,3-d]pyrimidin-5-yl)(piperidin-1-yl)methanone.

In other embodiments of the compound of formula (I), X is S, Y is S, and R₄ is t-butyl and the compound of formula (I) is:

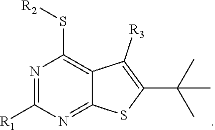

In particular embodiments, the compound of formula (I) is:

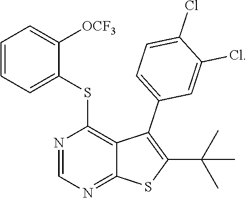 [SMILES: CC(C)(C)c1sc2ncnc(Sc3ccccc3OC(F)(F)F)c2c1-c1ccc(Cl)c(Cl)c1]

In other embodiments of the compound of formula (I), X is 0 and Y is 0 and the compound of formula (I) is:

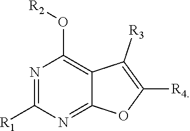

In yet other embodiments of the compound of formula (I), X is S and Y is 0 and the compound of formula (I) is:

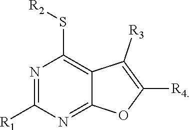

B. Methods for Treating Neuropathic Pain

In some embodiments, the presently disclosed subject matter provides a method for treating pain in a subject in need of treatment thereof, the method comprising administering to the subject a compound of formula (I), or a pharmaceutically acceptable salt thereof, in a therapeutically effective amount to treat the pain.

In some embodiments, the pain comprises neuropathic pain. Neuropathic pain can be caused by injury or infection of peripheral sensory nerves. Neuropathic pain includes, but is not limited to, pain from peripheral nerve trauma, herpes virus infection, diabetes mellitus, causalgia, plexus avulsion, neuroma, limb amputation, and vasculitis. Neuropathic pain also can be caused by nerve damage from chronic alcoholism, human immunodeficiency virus infection, hypothyroidism, uremia, or vitamin deficiencies. Stroke (spinal or brain) and spinal cord injury also can induce neuropathic pain. Cancer-related neuropathic pain results from tumor growth compression of adjacent nerves, brain, or spinal cord. Further, cancer treatments, including chemotherapy and radiation therapy, also can cause nerve injury. Accordingly, neuropathic pain can include, but is not limited to, chemotherapy-induced pain, post-traumatic injury pain, crush pain, painful traumatic mononeuropathy, painful polyneuropathy, pain resulting from spinal injury, nerve compression or entrapment, sacral pain, trigeminal neuralgia, migraine and migraine headache, postherpetic neuralgia, phantom limb pain, diabetic neuropathy, including diabetic peripheral neuropathic pain, postamputation pain, lumbar radiculopathy, and complex regional pain syndromes.

In some embodiments, the neuropathic pain comprises chronic neuropathic pain. Chronic neuropathic pain is a heterogeneous disease state with an unclear etiology. In chronic neuropathic pain, the pain can be mediated by multiple mechanisms. This type of pain generally arises from injury to the peripheral or central nervous tissue. The syndromes include pain associated with spinal cord injury, multiple sclerosis, post-herpetic neuralgia, trigeminal neuralgia, phantom pain, causalgia, and reflex sympathetic dystrophy and lower back pain, and other neuropathic pain described hereinabove. Chronic pain is different from acute pain in that patients suffer the abnormal pain sensations that can be described as spontaneous pain, continuous superficial burning and/or deep aching pain. The pain can be evoked by heat-, cold-, and mechano-hyperalgesia or by heat-, cold-, or mechano-allodynia.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

In some embodiments, the presently disclosed methods attenuate or alleviate pain. As used herein, the terms “attenuate” or “alleviate” intended to mean that the use of the presently disclosed compounds of formula (I) substantially reduces pain and the severity of any symptoms associated with pain.

In some embodiments, the method further comprises eliminating or attenuating one or more of off-target side effects, opioid-like side effects, and abuse potential. In certain embodiments, the off-target side effect comprises itching.

In some embodiments, the compound of formula (I) comprises a positive allosteric modulator of MRGPRX1. In certain embodiments, the MRGPRX1 is expressed in one or more DRG neurons.

In some embodiments, the presently disclosed method further comprises administering one or more additional therapeutic agents in combination with a compound of formula (I).

The term “combination” is used in its broadest sense and means that a subject is administered at least two agents, more particularly a compound of formula (I) and at least one additional therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds of formula (I) described herein can be administered alone or in combination with adjuvants that enhance stability of the compounds of formula (I), alone or in combination with one or more agents for treating pain, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound of formula (I) and at least one additional therapeutic agent can be varied so long as the beneficial effects of the combination of these agents are achieved. Accordingly, the phrase “in combination with” refers to the administration of a compound of formula (I) and at least one additional therapeutic agent either simultaneously, sequentially, or a combination thereof. Therefore, a subject administered a combination of a compound of formula (I) and at least one additional therapeutic agent can receive compound of formula (I) and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of formula (I) and at least one additional therapeutic agent are administered simultaneously, they can be administered to the subject as separate pharmaceutical compositions, each comprising either a compound of formula (I) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound of formula (I) and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In some embodiments, the one or more additional therapeutic agents are selected from the group consisting of a therapeutic agent for pain and an anti-inflammatory agent.

Examples of therapeutic agents known in the art to treat pain, including neuropathic pain, include, without limitation, opioid or non-opioid analgesic agents. Representative opioid analgesic agents include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene.

Representative non-opioid analgesic agents include, but are not limited to, aspirin, acetaminophen, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In other embodiments, the one or more therapeutic agents include a neuropathic disorder agent (e.g., pregabalin), an antidepressant (e.g., duloxetine, amitriptyline), a regional anesthetic (e.g., lidocaine), ketamine, and combinations thereof.

In some embodiments, the one or more therapeutic agents comprise an anti-inflammatory agent selected from the group consisting of a steroid (e.g., prednisolone), an antihistamine (e.g., loratadine), and combinations thereof.

In some embodiments, the compound of formula (I) is administered systemically. In particular embodiments, the systemic administration is selected from the group consisting of oral, buccal, sublingual, nasal, via an inhaler, suppository, topical, transdermal, intradermal, subcutaneous, intramuscular, intravenous, and intraperitoneal.

In some embodiments, the compound of formula (I) penetrates the central nervous system.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

C. Pharmaceutical Compositions and Administration

In another aspect, the present disclosure provides a pharmaceutical composition including one compound of formula (I) alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000).

In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hanks' solution, R₁ nger's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### II. Chemical Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁-20 inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

In particular embodiments, C₁-C₄ alkyl includes methyl, ethyl, n-propyl, isopropyl, n-butyl, isobutyl, sec-butyl, and tert-butyl.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁-8 alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁-8 straight-chain alkyls. In other embodiments, “alkyl” refers, in particular, to C₁-8 branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)— CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7 membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6 tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂-20 inclusive straight or branched hydrocarbon moiety having at least one carbon-carbon double bond by the removal of a single hydrogen molecule. Alkenyl groups include, for example, ethenyl (i.e., vinyl), propenyl, butenyl, 1 methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′- and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2 imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

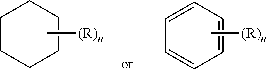

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

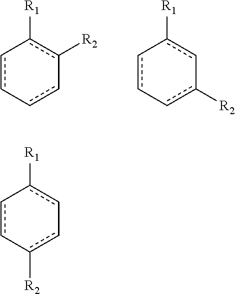

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′,—C(O)NR′R″, —OC(O)NR′R″, NR″C(O)R′, —NR′—C(O)NR″R′″, —NR″C(O)OR′, —NR—C(NR′R”)═NR′″, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R′″R′″)═NR″ ″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging . . . from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U-, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′— (C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁-20 inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″taken together may optionally be —(CH₂)ₖ— where k is an integer from 2 to 6.

Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —CN group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4 chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### General Procedures for the Synthesis of Representative Compounds

All solvents were reagent grade or HPLC grade. Unless otherwise noted, all materials were obtained from commercial suppliers and used without further purification. Compound A was purchased from Enamine. Compounds B and C were purchased from Key Organics. Synthesis of compounds D and E was previously reported in Chemica Sinica (2012), 3(1), 198-203. Compound 1a was purchased from LabNetwork. Melting points were obtained on a Mel-Temp apparatus and are uncorrected. ¹H NMR spectra were recorded at 400 MHz or 500 MHz. Chemical shifts are reported in parts per million relative to TMS. Preparative HPLC purification was performed on an Agilent 1200 series HPLC system equipped with an Agilent G1315D diode array detector using a Phenomenex Luna 5 μm C18(2) column (21.2 mm×250 mm, 5 μm).

The preparative HPLC solvent system consisted of distilled water and acetonitrile, both containing 0.1% formic acid. Preparative Method A used a flowrate of 15 mL/min with a gradient of 40% ACN/60% H₂O for 5 min followed by an increase to 100% ACN/0% H₂O over 40 minutes and a continuation of 100% ACN/0% H₂O until 50 minutes. Preparative Method B. used a flowrate of 15 mL/min with a gradient of 40% ACN/60% H₂O with an increase to 100% ACN/0% H₂O over 60 min and a continuation of 100% ACN/0% H₂O until 80 min. Preparative Method C used a flowrate of 15 mL/min with a gradient of 75% ACN/25% H₂O with an increase to 100% ACN/0% H₂O over 40 minutes and a continuation of 100% ACN/0% H₂O until 50 minutes. Preparative Method D used a flowrate of 15 mL/min with a gradient of 75% ACN/25% H₂O with an increase to 100% ACN/0% H₂O over 30 minutes and a continuation of 100% ACN/0% H₂O until 60 minutes.

Analytical HPLC was performed on an Agilent 1200 series HPLC system equipped with an Agilent G1315D diode array detector (detection at 220 nm) and an Agilent 6120 quadrupole MS detector. The HPLC solvent system consisted of distilled water and acetonitrile, both containing 0.1% formic acid. Analytical HPLC Mehotd A used a flowrate of 1.25 mL/min with a gradient of 20% ACN/80% H₂O for 0.25 minutes followed by an increase to 85% ACN/15% H₂O over 1.75 minutes and continuation of 85% ACN/15% H₂O until 4 minutes (detection at 220 nm) with an Eclipse Plus C18 column (3.5 micron, 2.1×50 mm). Analytical HPLC Method B used a flowrate of 0.75 mL/min with a gradient of 20% ACN/80% H₂O for 0.5 minutes followed by an increase to 85% ACN/15% H₂O over 4.5 minutes and continuation of 85% ACN/15% H₂O until 10 minutes (detection at 220 nm) with an Eclipse Plus C18 column (3.5 micron, 2.1×50 mm). Analytical HPLC Method C used a flowrate of 1.25 mL/min with a gradient of 5% ACN/95% H₂O for 0.25 minutes followed by an increase to 40% ACN/60% H₂O over 1.75 minutes and continuation of 40% ACN/60% H₂O until 4 minutes (detection at 220 nm) with an Eclipse Plus C18 column (3.5 micron, 2.1×50 mm). All final compounds tested were confirmed to be of >95% purity by the analytical HPLC methods described above unless otherwise noted.

### Example 2

### Synthetic Schemes for Representative Compounds

The presently disclosed active compounds may be prepared as described in the following reaction schemes.

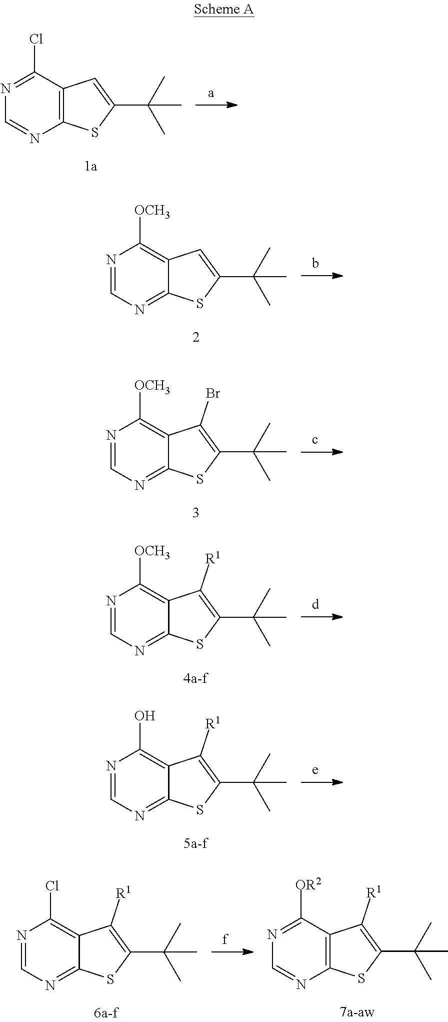

Reagents and conditions: (a) MeONa, MeOH, 0° C. to rt; (b) NBS, AcOH, 55° C.; (c) R¹B(OH)₂ PdCl₂(PPh₃)₂, K₂CO₃, DMF, 80° C.; (d) BBr₃, dichloromethane, rt; (e) POCl₃, 100° C.; (f) R²OH, NaH, DMF, 0° C. to rt.

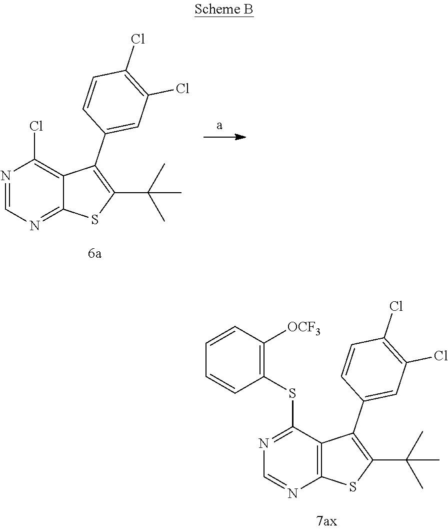 [SMILES: CC(C)(C)c1sc2ncnc(Cl)c2c1-c1ccc(Cl)c(Cl)c1 ; CC(C)(C)c1sc2ncnc(Sc3ccccc3OC(F)(F)F)c2c1-c1ccc(Cl)c(Cl)c1]

Reagents and conditions: (a) 2-(Trifluoromethoxy)thiophenol, NaH, DMF, 0° C. to rt.

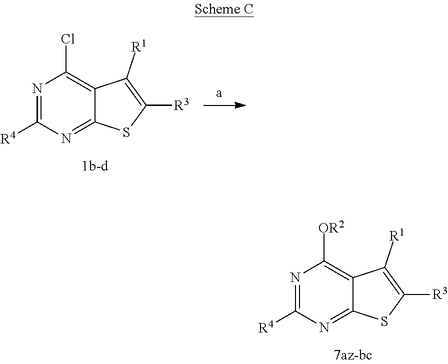

Reagents and conditions: (a) R²OH, NaH, DMF, 0° C. to rt.

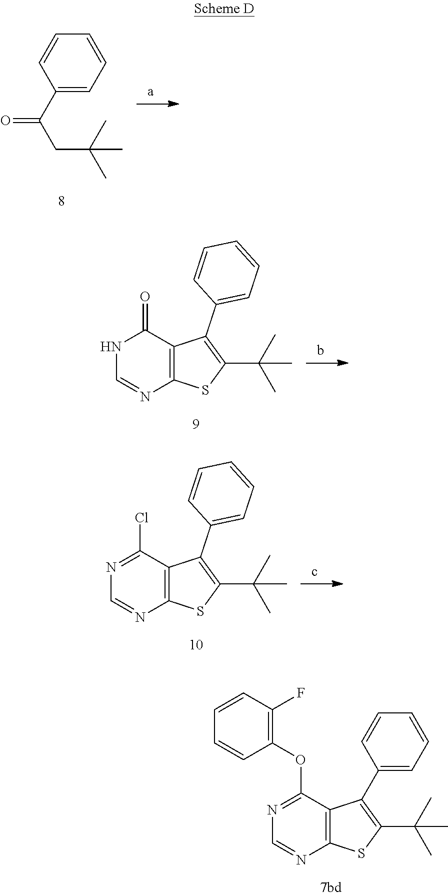 [SMILES: CC(C)(C)CC(=O)c1ccccc1 ; CC(C)(C)c1sc2nc[nH]c(=O)c2c1-c1ccccc1 ; CC(C)(C)c1sc2ncnc(Cl)c2c1-c1ccccc1 ; CC(C)(C)c1sc2ncnc(Oc3ccccc3F)c2c1-c1ccccc1]

Reagents and conditions: (a) ethyl cyanoacetate, S₈, formamide, diethylamine, L-proline, 170° C.; (b) POCl₃, 100° C.; (c) 2-fluorophenol, NaH, DMF, 0° C. to rt.

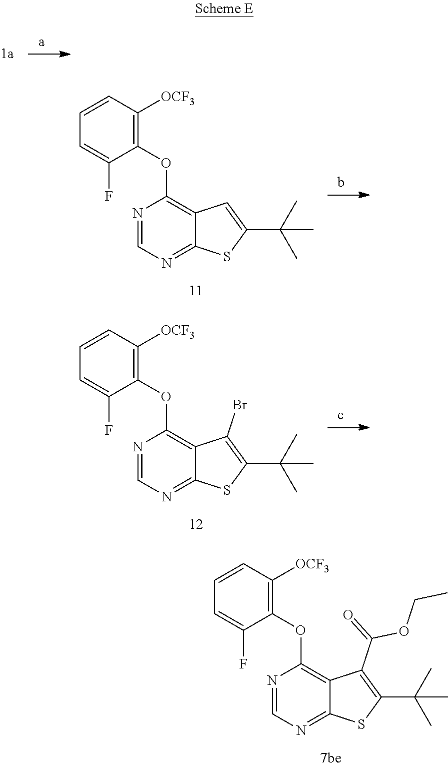 [SMILES: CC(C)(C)c1cc2c(Oc3c(F)cccc3OC(F)(F)F)ncnc2s1 ; CC(C)(C)c1sc2ncnc(Oc3c(F)cccc3OC(F)(F)F)c2c1Br ; CCOC(=O)c1c(C(C)(C)C)sc2ncnc(Oc3c(F)cccc3OC(F)(F)F)c12]

Reagents and conditions: (a) NaH, DMF, 2-fluoro-6-(trifluoromethoxy)phenol, 60° C., (b) NBS, AcOH 55° C.; (c) n-BuLi, ethyl chloroformate, THF, −78° C.

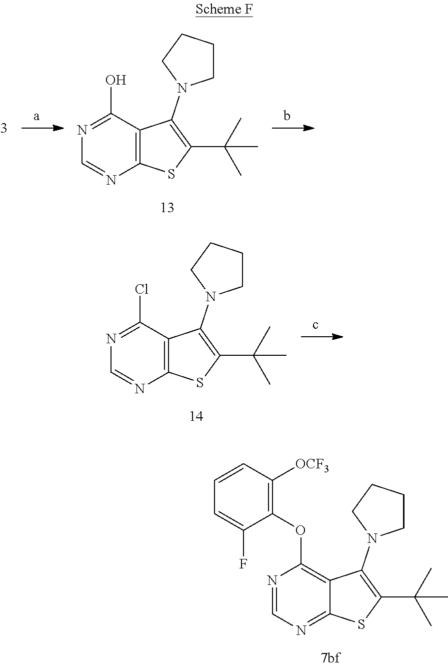 [SMILES: CC(C)(C)c1sc2ncnc(O)c2c1N1CCCC1 ; CC(C)(C)c1sc2ncnc(Cl)c2c1N1CCCC1 ; CC(C)(C)c1sc2ncnc(Oc3c(F)cccc3OC(F)(F)F)c2c1N1CCCC1]

Reagents and conditions: (a) pyrrolidine, Cu₂O, water, 120° C.; (b) POCl₃, 100° C.; (c) 2-fluoro-6-(trifluoromethoxy)phenol, NaH, DMF, 0° C. to rt.

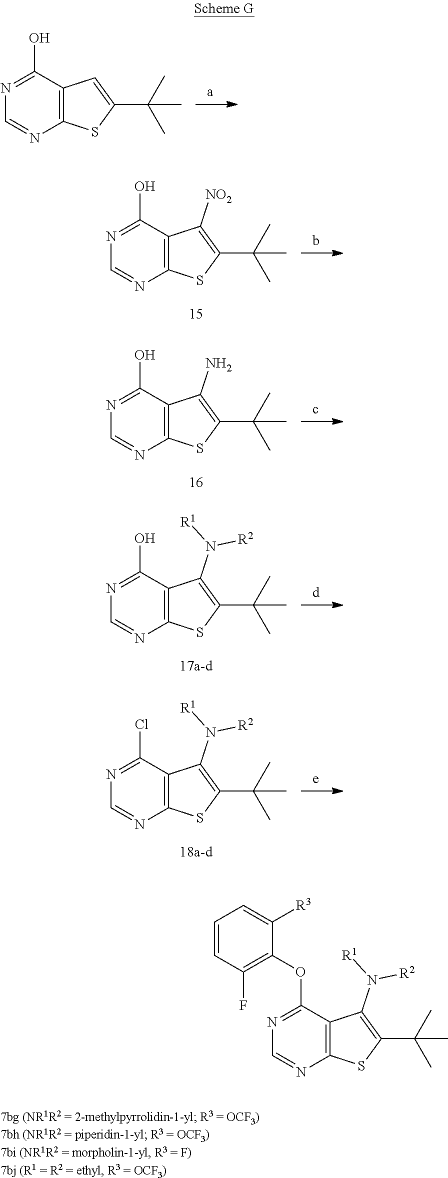

Reagents and conditions: (a) HNO₃, H₂SO₄, 0° C. to rt; (b) Zn dust, MeOH/DCM, sat. NH₄Cl, rt; (c) Aldehydes, NaCNBH₃, AcOH, MeOH, rt; (d) POCl₃, 110° C.; (e) ArOH, NaH, DMF, 0° C. to rt.

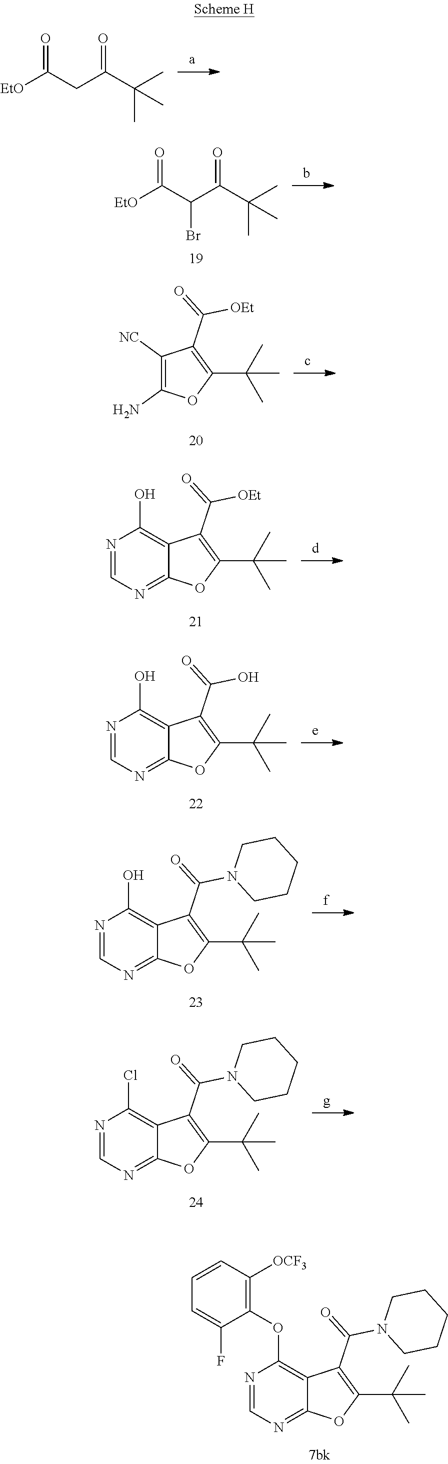

Reagents and conditions: (a) NBS, DMSO, rt; (b) malononitrile, NaOEt, EtOH, rt; (c) formic acid, acetic anhydride, 100° C.; (d) 6M HCl, reflux; (e) HATU, DIEA, piperidine, DMF, rt; (f) POCl₃, 110° C.; (g) NaH, DMF, 2-fluoro-6-(trifluoromethoxy)phenol, 60° C.

### Example 3

### Synthesis of 6-tert-butyl-5-(3,4-dichlorophenyl)-4-(2-(trifluoromethoxy)phenoxy)thieno[2,3-d]pyrimidine (7a)

### 6-tert-Butyl-4-methoxythieno[2,3-d]pyrimidine (2)

To a solution of 6-tert-butyl-4-chlorothieno[2,3-d]pyrimidine 1a (1.62 g, 7.15 mmol) in methanol (20 mL) at 0° C. was added 25 wt. % sodium methoxide in methanol (17.2 mmol, 3.92 mL). The mixture was stirred at 0° C. for 30 min, then at rt for 1 h. The solvent was removed in vacuo and water was carefully added to quench excess of sodium methoxide. The compound was extracted with EtOAc and the organic layer was dried over Na₂SO₄ and concentrated to give 1.56 g (98% crude yield) of 2 as an oil which was used without further purification. ¹H NMR (CDCl₃): δ 1.45 (s, 9H), 4.12 (s, 3H), 7.06 (s, 1H), 8.59 (s, 1H).

### 5-Bromo-6-tert-butyl-4-methoxythieno[2,3-d]pyrimidine (3)

To a solution of 2 (1.56 g, 7.02 mmol) in AcOH (30 mL) was added N bromosuccinimide (3.75 g, 21.1 mmol) and the mixture was stirred at 55° C. overnight. The reaction was concentrated, and the crude material was purified by Biotage Isolera One using 0-10% EtOAc in hexanes as eluent to give 1.8 g (85%) of 3 as a light yellow solid. ¹H NMR (CDCl₃): δ 1.59 (s, 9H), 4.14 (s, 3H), 8.59 (s, 1H).

### 6-tert-Butyl-5-(3,4-dichlorophenyl)-4-methoxythieno[2,3-d]pyrimidine (4a)

A suspension of 3 (3.00 g, 9.96 mmol), 3,4-dichlorophenylboronic acid (3.80 g, 19.9 mmol), potassium carbonate (4.13 g, 29.9 mmol), and bis(triphenylphosphine)palladium(II) dichloride (0.699 g, 1.00 mmol) in DMF (45 mL) was purged with N2 gas via an inserted long needle for 30 min. The mixture was then stirred at 80° C. overnight. After removing solvent in vacuo, water was added to the mixture. The mixture was extracted with dichloromethane twice and the combined extracts were washed with brine, dried over sodium sulfate, and concentrated.

Resulting white precipitate was removed by filtration and the filtrate was concentrated and purified by Biotage Isolera One using 5% EtOAc/DCM as eluent to give 2.73 g (75%) of compound 4a as a thick brown oil. ¹H NMR (CDCl₃): δ 1.30 (s, 9H), 3.74 (s, 3H), 7.13 (dd, J=2.0, 8.1 Hz, 1H), 7.40 (d, J=2.0 Hz, 1H), 7.45 (d, J=8.3 Hz, 1H), 8.58 (s, 1H). HPLC (Method A): retention time 2.61 min, m/z 367.1 [M+H]⁺.

### 6-tert-Butyl-5-(3,4-dichlorophenyl)thieno[2,3-d]pyrimidin-4-ol (5a)

To a solution of 4a (1.50 g, 4.08 mmol) in DCM (50 mL) at 0° C. was added 1 M BBr₃ solution in DCM (20.4 mL, 20.4 mmol). The mixture was stirred at 0° C., then gradually warmed to rt and stirred until completion (7 days). The mixture was then cooled to 0° C. and methanol was added to quench excess of BBr₃. After removal of solvents, the crude material was partitioned between water and dichloromethane. The organic layer was washed with brine, dried over sodium sulfate, and concentrated. The residual solid was triturated with dichloromethane to give 1.27 g (93%) of 5a as a white solid. ¹H NMR (CDCl₃): δ 1.28 (s, 9H), 7.14 (dd, J=2.0, 8.1 Hz, 1H), 7.40 (d, J=2.0 Hz, 1H), 7.46 (d, J=8.3 Hz, 1H), 7.76 (s, 1H), 11.25 (s, 1H). Mp>250° C.; HPLC (Method A): retention time 2.23 min, m/z 353.1 [M+H]⁺.

### 6-tert-Butyl-4-chloro-5-(3,4-dichlorophenyl)thieno[2,3-d]pyrimidine (6a)

A solution of 5a (2.00 g, 5.66 mmol) in POCl₃ (15 mL) was stirred at 110° C. for 1 h. Excess POCl₃ was removed in vacuo followed by co-evaporation with DCM (three times). The residual material was passed through a short silica column using 0-10% EtOAc in DCM as eluent to give 2.00 g (95%) of 6a as a white solid. ¹H NMR (CDCl₃): δ 1.33 (s, 9H), 7.17 (dd, J=2.0, 8.3 Hz, 1H), 7.45 (d, J=2.0 Hz, 1H), 7.51 (d, J=8.1 Hz, 1H), 7.79 (s, 1H).

### 6-tert-Butyl-5-(3,4-dichlorophenyl)-4-(2-(trifluoromethoxy)phenoxy)thieno[2,3 d]pyrimidine (7a)

To a solution of 2-trifluoromethoxyphenol (0.529 g, 2.97 mmol) in DMF (6 mL) was added sodium hydride (60% dispersion in mineral oil, 0.119 g, 2.97 mmol) at 0° C. and the mixture was stirred at 0° C. for 5-10 min. A solution of 6a (0.920 g, 2.48 mmol) in DMF (15 mL) was then slowly added to the mixture via syringe at 0° C. The reaction mixture was gradually warmed up to rt and stirred overnight. A few drops of 10% KHSO₄ was added to the mixture to quench excess of NaH and the mixture was concentrated in vacuo. The crude material was partitioned between water and EtOAc. The organic layer was washed with brine, dried over sodium sulfate, and concentrated. The residual material was purified by Biotage Isolera One using CHCl₃ as eluent to give 0.880 g (69%) of 7a as a white solid. Mp 85-89° C. ¹H NMR (CDCl3): δ 1.35 (s, 9H), 7.02 (m, 1H), 7.23-7.33 (m, 4H), 7.43 (d, J=8.3 Hz, 1H), 7.51 (d, J=2.0 Hz, 1H), 8.51 (s, 1H). ¹³C NMR (CDCl₃): δ 32.6, 36.5, 119.9, 120.2 (q, J_{C-F}=259 Hz), 122.4, 124.1, 126.2, 126.7, 127.7, 129.5, 129.9, 131.6, 131.8, 132.2, 137.0, 140.9, 143.7, 152.3, 152.4, 162.1, 166.0. Anal. Calcd. for C₂₃H₁₇C₁₂F₃N₂O₂S: C, 53.81; H, 3.34; N, 5.46; S, 6.25; F, 11.10; Cl, 13.81. Found: C, 53.82; H, 3.41; N, 5.38, S, 6.23; F, 10.93; Cl, 13.87. HPLC (Method A): >99% by area, retention time 3.04 min, m/z 513.1 [M+H]⁺.

### Example 4

### Synthesis of 6-tert-butyl-5-(3,4-dichlorophenyl)-4-(2-fluoro-6-(trifluoromethoxy)phenoxy)thieno[2,3-d]pyrimidine (7b)

Compound 7b was prepared as described for the preparation of compound 7a with the exception that 2-fluoro-6-(trifluoromethoxy)phenol was used in place of 2-(trifluoromethoxy)phenol. The crude material was triturated in a mixture of methanol and water to afford 7b as a white solid (70% yield). Mp 134-137° C.; ¹H NMR (CDCl₃): δ 1.35 (s, 9H), 7.13 (m, 2H), 7.22 (m, 2H), 7.43 (m, 1H), 7.51 (m, 1H), 8.50 (s, 1H). HPLC (Method A): retention time 3.16 min, m/z 531.1 [M+H]⁺.

### Example 5

### Synthesis of 6-tert-butyl-4-(2-chloro-6-(trifluoromethoxy)phenoxy)-5-(3,4-dichlorophenyl)thieno[2,3-d]pyrimidine (7c)

Compound 7c was prepared as described for the preparation of compound 7a with the exception that 2-chloro-6-(trifluoromethoxy)phenol was used in place of 2-(trifluoromethoxy)phenol. The crude material was triturated in a mixture of methanol and water to afford 7c as a white solid (61% yield). Mp 66° C.; ¹H NMR (CDCl₃): δ 1.35 (s, 9H), 7.23 (m, 2H), 7.29 (m, 1H), 7.37 (m, 1H), 7.44 (m, 1H), 7.49 (m, 1H), 8.48 (s, 1H). HPLC (Method A): retention time 3.23 min, m/z 547.0 [M+H]⁺.

### Example 6

### Synthesis of 6-tert-butyl-5-(3,4-dichlorophenyl)-4-(3-(trifluoromethoxy)phenoxy)thieno[2,3-d]pyrimidine (7d)

Compound 7d was prepared as described for the preparation of compound 7a with the exception that 3-(trifluoromethoxy)phenol was used in place of 2-(trifluoromethoxy)phenol. The crude material was triturated in a mixture of methanol and water to afford 7d as a white solid (68% yield). Mp 121° C.; ¹H NMR (CDCl₃): δ 1.35 (s, 9H), 6.82 (m, 1H), 6.87 (m, 1H), 7.05 (m, 1H), 7.23 (dd, J=1.8, 8.1 Hz, 1H), 7.36 (t, J=8.1 Hz, 1H), 7.46 (d, J=8.1 Hz, 1H), 7.52 (d, J=1.8 Hz, 1H), 8.57 (s, 1H). HPLC (Method A): retention time 3.00 min, m/z 513.1 [M+H]⁺.

### Example 7

### Synthesis of 6-tert-butyl-5-(3,4-dichlorophenyl)-4-(4-(trifluoromethoxy)phenoxy)thieno[2,3-d]pyrimidine (7e)

Compound 7e was prepared as described for the preparation of compound 7a with the exception that 4-(trifluoromethoxy)phenol was used in place of 2-(trifluoromethoxy)phenol. The crude material was triturated in a mixture of methanol and water to afford 7e as a white solid (80% yield). Mp 103° C.; ¹H NMR (CDCl₃): δ 1.35 (s, 9H), 6.93 (d, J=9.1 Hz, 1H), 7.18 (d, J=8.6 Hz, 2H), 7.24 (dd, J=1.8, 8.1 Hz, 1H), 7.45 (d, J=8.1 Hz, 1H), 7.52 (d, J=1.8 Hz, 1H), 8.56 (s, 1H). HPLC (Method A): retention time 2.97 min, m/z 513.2 [M+H]⁺.

### Example 8

### Synthesis of 6-tert-butyl-5-(3,4-dichlorophenyl)-4-(4-nitro-2-(trifluoromethoxy)phenoxy)thieno[2,3-d]pyrimidine (7f)

Compound 7f was prepared as described for the preparation of compound 7a with the exception that 4-nitro-2-(trifluoromethoxy)phenol was used in place of 2 (trifluoromethoxy)phenol. The product was purified by Biotage Isolera One using 20-50% EtOAc/hexanes as eluent to give 7f as colorless semi-solid (63% yield). ¹H NMR (CDCl₃): δ 1.36 (s, 9H), 7.22-7.26 (m, 2H), 7.43 (d, J=8.1 Hz, 1H), 7.50 (d, J=2.0 Hz, 1H), 8.21 (m, 2H), 8.49 (s, 1H). HPLC (Method A): retention time 2.91 min, m/z 558.1 [M+H]⁺.

### Example 9

### Synthesis of 6-tert-butyl-5-(3,4-dichlorophenyl)-4-(2-fluoropyridin-3-yloxy)thieno[2,3-d]pyrimidine (7g)

Compound 7g was prepared as described for the preparation of compound 7a with the exception that 2-fluoropyridin-3-ol was used in place of 2-(trifluoromethoxy)phenol. The crude product was purified by preparative HPLC (Method B) to afford 7g as a white solid (42% yield). Mp 164° C.; ¹H NMR (CDCl₃): δ 1.35 (s, 9H), 7.19 (m, 1H), 7.26 (m, 2H), 7.45 (d, J=8.1 Hz, 1H), 7.54 (d, J=1.8 Hz, 1H), 8.51 (s, 1H). HPLC (Method A): retention time 2.68 min, m/z 448.2 [M+H]⁺.

### Example 10

### Synthesis of 6-tert-butyl-5-(3,4-dichlorophenyl)-4-(2-(trifluoromethoxy)pyridin-3-yloxy)thieno[2,3-d]pyrimidine (7h)

Compound 7h was prepared as described for the preparation of compound 7a with the exception that 2-(trifluoromethoxy)pyridin-3-ol was used in place of 2-(trifluoromethoxy)phenol. The crude material was triturated in a mixture of methanol and water to afford 7h as a grey solid (48% yield). Mp 117° C.; ¹H NMR (CDCl₃): δ 1.36 (s, 9H), 7.25 (m, 2H), 7.45 (m, 2H), 7.51 (d, J=2.0 Hz, 1H), 8.17 (dd, J=1.5, 4.8 Hz, 1H), 8.50 (s, 1H). HPLC (Method A): retention time 2.81 min, m/z 514.1 [M+H]⁺.

### Example 11

### Synthesis of 6-tert-butyl-5-(3,4-dichlorophenyl)-4-(2-methoxypyridin-3-yloxy)thieno[2,3-d]pyrimidine (7i)

Compound 7i was prepared as described for the preparation of compound 7a with the exception that 2-methoxypyridin-3-ol was used in place of 2 (trifluoromethoxy)phenol. The crude material was triturated in a mixture of methanol and water to afford 7i as a beige solid (57% yield). Mp 173° C.; ¹H NMR (CDCl₃): δ 1.35 (s, 9H), 3.85 (s, 2H), 6.87 (dd, J=5.3, 7.8 Hz, 1H), 7.22 (m, 1H), 7.29 (m, 1H), 7.42 (d, J=8.1 Hz, 1H), 7.54 (d, J=1.8 Hz, 1H), 8.01 (dd, J=1.5, 5.1 Hz, 1H), 8.50 (s, 1H). HPLC (Method A): retention time 2.69 min, m/z 460.1 [M+H]⁺.

### Example 12

### Synthesis of 3-(6-tert-butyl-5-(3,4-dichlorophenyl)thieno[2,3-d]pyrimidin-4-yloxy)pyridin-2-amine (7j)

Compound 7j was prepared as described for the preparation of compound 7a with the exception that 2-aminopyridin-3-ol was used in place of 2-(trifluoromethoxy)phenol. The crude material was purified by preparative HPLC (Method A) to afford 7j (22% yield as a white solid. Mp 207° C.; ¹H NMR (CDCl₃): δ 1.35 (s, 9H), 4.14 (s, 2H), 6.68 (dd, J=48, 7.8 Hz, 1H), 7.26-7.30 (m, 2H), 7.49 (d, J=8.1 Hz, 1H), 7.55 (d, J=2.0 Hz, 1H), 7.92 (dd, J=1.5, 4.8 Hz, 1H), 8.57 (s, 1H). HPLC (Method A): retention time 1.95 min, m/z 445.2 [M+H]⁺.

### Example 13

### Synthesis of 6-tert-butyl-5-(3,4-dichlorophenyl)-4-(pyridazin-4-yloxy)thieno[2,3-d]pyrimidine (7k)

Compound 7k was prepared as described for the preparation of compound 7a with the exception that pyridazin-4-ol was used in place of 2-(trifluoromethoxy)phenol. The crude material was purified by preparative HPLC (Method A) to afford 7k as a beige solid (69% yield). Mp 186° C.; ¹H NMR (CDCl₃): δ 1.36 (s, 9H), 6.40 (m, 1H), 7.04 (m, 1H), 7.20 (m, 1H), 7.29 (m, 1H), 7.32 (m, 1H), 8.14 (dd, J=1.8, 8.1 Hz, 1H), 8.98 (s, 1H). HPLC (Method A): retention time 2.06 min, m/z 431.1 [M+H]⁺.

### Example 14

### Synthesis of 6-tert-butyl-5-(3,4-dichlorophenyl)-4-phenoxythieno[2,3-d]pyrimidine (7l)

Compound 71 was prepared as described for the preparation of compound 7a with the exception that phenol was used in place of 2-(trifluoromethoxy)phenol. The crude material was triturated in a mixture of methanol and water to afford 7l as a beige solid (78% yield). Mp 148° C.; ¹H NMR (CDCl₃): δ 1.35 (s, 9H), 6.91 (d, J=8.6 Hz, 2H), 7.19-7.26 (m, 2H), 7.33 (t, J=8.1 Hz, 2H), 7.44 (d, J=8.1 Hz, 1H), 7.53 (d, J=2.0 Hz, 1H), 8.55 (s, 1H). HPLC (Method A): retention time 2.77 min, m/z 429.1 [M+H]⁺

### Example 15

### Synthesis of 6-tert-butyl-5-(3,4-dichlorophenyl)-4-(2-fluorophenoxy)thieno[2,3-d]pyrimidine (7m)

Compound 7m was prepared as described for the preparation of compound 7a with the exception that 2-fluorophenol was used in place of 2-(trifluoromethoxy)phenol. The crude material was triturated in a mixture of methanol and water to afford 7m as a beige solid (80% yield). Mp 163° C.; ¹H NMR (CDCl₃): δ 1.35 (s, 9H), 6.99 (m, 1H), 7.13-7.19 (m, 3H), 7.30 (m, 21H), 7.44 (d, J=8.3 Hz, 1H), 7.54 (d, J=2.0 Hz, 1H), 8.53 (s, 1H). HPLC (Method A): retention time 2.82 min, m/z 447.1 [M+H]⁺.

### Example 16

### Synthesis of 6-tert-butyl-5-(3,4-dichlorophenyl)-4-(2,6-difluorophenoxy)thieno[2,3-d]pyrimidine (7n)

Compound 7n was prepared as described for the preparation of compound 7a with the exception that 2,6-difluorophenol was used in place of 2-(trifluoromethoxy)phenol. The crude material was triturated in a mixture of methanol and water to afford 7n as a beige solid (78% yield). Mp 205° C.; ¹H NMR (CDCl₃): δ 1.35 (s, 9H), 6.93 (t, J=8.3 Hz, 2H), 7.14 (m, 1H), 7.27 (dd, J=2.0, 8.1 Hz, 1H), 7.45 (d, J=8.1 Hz, 1H), 7.56 (d, J=2.0 Hz, 1H), 8.53 (s, 1H). HPLC (Method A): retention time 2.78 min, m/z 465.1 [M+H]⁺.

### Example 17

### Synthesis of 6-tert-butyl-5-(3,4-dichlorophenyl)-4-(3-fluorophenoxy)thieno[2,3-d]pyrimidine (70)

Compound 7o was prepared as described for the preparation of compound 7a with the exception that 3-fluorophenol was used in place of 2 (trifluoromethoxy)phenol. The crude material was triturated in a mixture of methanol and water to afford 7o as a white solid (95% yield). Mp 144° C.; ¹H NMR (CDCl₃): δ 1.35 (s, 9H), 6.70 (m, 2H), 6.92 (m, 1H), 7.23 (dd, J=2.0, 8.1 Hz, 1H), 7.29 (m, 1H), 7.45 (d, J=8.1 Hz, 1H), 7.51 (d, J=2.0 Hz, 1H), 8.57 (s, 1H). HPLC (Method A): retention time 2.76 min, m/z 447.1 [M+H]⁺.

### Example 18

### Synthesis of 6-tert-butyl-5-(3,4-dichlorophenyl)-4-(4-fluorophenoxy)thieno[2,3-d]pyrimidine (7p)

Compound 7p was prepared as described for the preparation of compound 7a with the exception that 4-fluorophenol was used in place of 2-(trifluoromethoxy)phenol. The crude material was triturated in a mixture of methanol and water to afford 7p as a white solid (83% yield). Mp 179° C.; ¹H NMR (CDCl₃): δ 1.35 (s, 9H), 6.88 (m, 2H), 7.03 (m, 2H), 7.24 (dd, J=2.0, 8.1 Hz, 1H), 7.45 (d, J=8.1 Hz, 1H), 7.52 (m, 1H), 8.55 (s, 1H). HPLC (Method A): retention time 2.73 min, m/z 447.1 [M+H]⁺

### Example 19

### Synthesis of 6-tert-butyl-5-(3,4-dichlorophenyl)-4-(2-methoxyphenoxy)thieno[2,3-d]pyrimidine (7q)

Compound 7q was prepared as described for the preparation of compound 7a with the exception that 2-methoxyphenol was used in place of 2-(trifluoromethoxy)phenol. The crude material was triturated in a mixture of methanol and water to afford 7q as a beige solid (65% yield). Mp 136° C.; ¹H NMR (CDCl₃): δ 1.35 (s, 9H), 3.70 (s, 3H), 6.91-6.96 (m, 3H), 7.17 (m, 1H), 7.28 (m, 1H), 7.40 (d, J=8.3 Hz, 1H), 7.54 (d, J=2.0 Hz, 1H), 8.50 (s, 1H). HPLC (Method A): retention time 2.70 min, m/z 459.1 [M+H]⁺.

### Example 20

### Synthesis of 6-tert-butyl-5-(3,4-dichlorophenyl)-4-(2-ethoxyphenoxy)thieno[2,3-d]pyrimidine (7r)

Compound 7r was prepared as described for the preparation of compound 7a with the exception that 2-ethoxyphenol was used in place of 2 (trifluoromethoxy)phenol. The crude material was triturated in a mixture of methanol and water to afford 7r as a beige solid (55% yield). Mp 119° C.; ¹H NMR (CDCl₃): δ 1.15 (t, J=6.8 Hz, 3H), 1.35 (s, 9H), 3.90 (qt, J=7.1, 13.9 Hz, 2H), 6.89-6.95 (m, 3H), 7.14 (m, 1H), 7.25 (m, 1H), 7.40 (d, J=8.3 Hz, 1H), 7.54 (d, J=2.0 Hz, 1H), 8.50 (s, 1H). HPLC (Method A): retention time 2.69 min, m/z 473.2 [M+H]⁺.

### Example 21

### Synthesis of 6-tert-butyl-5-(3,4-dichlorophenyl)-4-(2-(2,2,2-trifluoroethoxy)phenoxy)thieno[2,3-d]pyrimidine (7s)

Compound 7s was prepared as described for the preparation of compound 7a with the exception that 2-(2,2,2-trifluoroethoxy)phenol was used in place of 2-(trifluoromethoxy)phenol. The crude material was triturated in a mixture of methanol and water to afford 7s as a grey solid (69% yield). Mp 136° C.; ¹H NMR (CDCl₃): δ 1.35 (s, 9H), 4.18 (m, 2H), 6.97 (m, 2H), 6.38 (m, 1H), 7.22 (m, 1H), 7.31 (m, 1H), 7.40 (m, 1H), 7.54 (m, 1H), 8.49 (s, 1H). HPLC (Method A): retention time 2.80 min, m/z 527.1 [M+H]⁺.

### Example 22

### Synthesis of 6-tert-butyl-5-(3,4-dichlorophenyl)-4-(o-tolyloxy)thieno[2,3-d]pyrimidine (7t)

Compound 7t was prepared as described for the preparation of compound 7a with the exception that o-cresol was used in place of 2-(trifluoromethoxy)phenol. The crude material was triturated in a mixture of methanol and water to afford 7t as a white solid (84% yield). Mp 151° C.; ¹H NMR (CDCl₃): δ 1.35 (s, 9H), 1.94 (s, 3H), 6.89-6.85 (d, J=7.6 Hz, 1H), 7.14-7.22 (m, 3H), 7.26 (m, 1H), 7.43 (d, J=8.1 Hz, 1H), 7.54 (d, J=1.8 Hz, 1H), 8.52 (s, 1H). HPLC (Method A): retention time 2.91 min, m/z 443.1 [M+H]⁺.

### Example 23

### Synthesis of 1-(2-(6-tert-butyl-5-(3,4-dichlorophenyl)thieno[2,3-d]pyrimidin-4-yloxy)phenyl)ethanone (7u)

Compound 7u was prepared as described for the preparation of compound 7a with the exception that 1-(2-hydroxyphenyl)ethanone was used in place of 2 (trifluoromethoxy)phenol. The crude material was purified by preparative HPLC (Method A) to afford 7u as a white solid (83% yield. Mp 60° C. ¹H NMR (CDCl₃): δ 1.34 (s, 9H), 2.33 (s, 3H), 6.88 (dd, J=1.0, 8.1 Hz, 1H), 7.26-7.33 (m, 2H), 7.41 (d, J=8.1 Hz, 1H), 7.49 (m, 1H), 7.53 (d, J=2.0 Hz, 1H), 7.77 (dd, J=1.8, 7.8 Hz, 1H), 8.48 (s, 1H). HPLC (Method A): retention time 2.66 min, m/z 471.2 [M+H]⁺.

### Example 24

### Synthesis of 2-(6-tert-butyl-5-(3,4-dichlorophenyl)thieno[2,3-d]pyrimidin-4-yloxy)benzonitrile (7v)

Compound 7v was prepared as described for the preparation of compound 7a with the exception that 2-hydroxybenzonitrile was used in place of 2-(trifluoromethoxy)phenol. The crude material was purified by preparative HPLC (Method A) to afford 7v as a semi-solid (4% yield). ¹H NMR (CDCl₃): δ 1.35 (s, 9H), 7.09 (d, J=8.3 Hz, 1H), 7.43 (m, 2H), 7.48 (d, J=8.3 Hz, 1H), 7.57 (d, J=2.0 Hz, 1H), 7.62-7.66 (m, 2H), 8.52 (s, 1H). HPLC (Method A): retention time 2.67 min, m/z 454.1 [M+H]⁺.

### Example 25

### Synthesis of 6-tert-butyl-5-(3,4-dichlorophenyl)-4-isopropoxythieno[2,3-d]pyrimidine (7w)

Compound 7w was prepared as described for the preparation of compound 7a with the exception that 2-propanol was used in place of 2-(trifluoromethoxy)phenol. The product was purified by Biotage Isolera One using 10-20% EtOAc/hexanes as eluent to give 7w as a beige solid (32% yield). Mp 165° C.; ¹H NMR (CDCl₃): δ 0.97 (m, 6H), 1.32 (s, 9H), 5.22 (m, 1H), 7.13 (dd, J=2.0, 8.1 Hz, 1H), 7.40 (d, J=2.0 Hz, 1H), 7.45 (d, J=8.3 Hz, 1H), 8.53 (s, 1H). HPLC (Method A): retention time 3.01 min, m/z 395.1 [M+H]⁺.

### Example 26

### Synthesis of 6-tert-butyl-4-cyclopropoxy-5-(3,4-dichlorophenyl)thieno[2,3-d]pyrimidine (7x)

Compound 7x was prepared as described for the preparation of compound 7a with the exception that cyclopropanol was used in place of 2 (trifluoromethoxy)phenol. The crude material was triturated in a mixture of methanol and water to afford 7x as a beige solid (25% yield). Mp 172° C.; ¹H NMR (CDCl₃): δ 0.22 (m, 2H), 0.64 (m, 2H), 1.31 (s, 9H), 4.35 (m, 1H), 7.09 (dd, J=2.0, 8.3 Hz, 1H), 7.38 (d, J=2.0 Hz, 1H), 7.45 (d, J=8.1 Hz, 1H), 8.63 (s, 1H). HPLC (Method A): retention time 2.74 min, m/z 393.1 [M+H]⁺.

### Example 27

### Synthesis of 6-tert-butyl-4-cyclobutoxy-5-(3,4-dichlorophenyl)thieno[2,3-d]pyrimidine (7y)

Compound 7y was prepared as described for the preparation of compound 7a with the exception that cyclobutanol was used in place of 2-(trifluoromethoxy)phenol. The crude material was triturated in a mixture of methanol and water to afford 7y as a beige solid (82% yield). Mp 146° C.; ¹H NMR (CDCl₃): δ 1.33 (s, 9H), 1.60 (m, 4H), 2.25 (m, 2H), 5.13 (m, 1H), 7.16 (dd, J=1.8, 8.3 Hz, 1H), 7.43 (d, J=2.0 Hz, 1H), 7.48 (d, J=8.3 Hz, 1H), 8.52 (s, 1H). HPLC (Method A): retention time 2.96 min, m/z 407.1 [M+H]⁺.

### Example 28

### Synthesis of 6-tert-butyl-4-(cyclopentyloxy)-5-(3,4-dichlorophenyl)thieno[2,3-d]pyrimidine (7z)

Compound 7z was prepared as described for the preparation of compound 7a with the exception that cyclopentanol was used in place of 2-(trifluoromethoxy)phenol. The crude material was triturated in a mixture of methanol and water to afford 7z as a white solid (44% yield). Mp 112° C.; ¹H NMR (CDCl₃): δ 1.29 (m, 2H), 1.31 (s, 9H), 1.46 (m, 3H), 1.68 (m, 1H), 1.70 (m, 2H), 5.41 (m, 1H), 7.13 (dd, J=2.0, 8.1 Hz, 1H), 7.41 (d, J=2.0 Hz, 1H), 7.45 (d, J=8.1 Hz, 1H), 8.55 (s, 1H). HPLC (Method A): retention time 3.20 min, m/z 421.1 [M+H]⁺.

### Example 29

Synthesis of 6-tert-butyl-4-(cyclohexyloxy)-5-(3,4-dichlorophenyl)thieno[2,3-d]pyrimidine (7aa)

Compound 7aa was prepared as described for the preparation of compound 7a with the exception that cyclohexanol was used in place of 2 (trifluoromethoxy)phenol. The crude material was triturated in a mixture of methanol and water to afford 7aa as a light yellow solid (37% yield). Mp 121° C.; ¹H NMR (CDCl₃): δ 1.16-1.44 (m, 15H), 1.64 (m, 4H), 5.13 (m, 1H), 7.15 (dd, J=2.0, 8.1 Hz, 1H), 7.43 (d, J=1.8 Hz, 1H), 7.46 (d, J=8.1 Hz, 1H), 8.53 (s, 1H). HPLC (Method A): retention time 3.78 min, m/z 435.2 [M+H]⁺.

### Example 30

### Synthesis of 6-tert-butyl-5-(3,4-dichlorophenyl)-4-(1-methylpiperidin-3-yloxy)thieno[2,3-d]pyrimidine (7ab)

Compound 7ab was prepared as described for the preparation of compound 7a with the exception that 1-methylpiperidin-3-ol was used in place of 2-(trifluoromethoxy)phenol. The crude material was purified by preparative HPLC (Method A) to afford 7ab as an oil (30% yield). ¹H NMR (CDCl₃): δ 1.32 (s, 9H), 1.57-1.64 (m, 2H), 1.82-1.93 (m, 2H), 2.12-2.25 (m, 2H), 2.33 (d, J=5.6 Hz, 3H), 2.71-2.89 (m, 2H), 5.12 (m, 1H), 7.13-7.20 (m, 1H), 7.40 (m, 1H), 7.47 (m, 1H), 8.53 (s, 1H). HPLC (Method A): retention time 1.76 min, m/z 450.2 [M+H]⁺.

### Example 31

### Synthesis of 6-tert-butyl-5-(3,4-dichlorophenyl)-4-(2-methylcyclohexyloxy)thieno[2,3-d]pyrimidine (7ac)

Compound 7ac was prepared as described for the preparation of compound 7a with the exception that 2-methylcyclohexanol was used in place of 2 (trifluoromethoxy)phenol. The crude material was purified by preparative HPLC (Method C) to afford 1:1 mixture of diastereomers 7ac as a light yellow oil (19% yield). ¹H NMR (CDCl₃): δ 0.60 and 0.69 (d, J=6.3 Hz, 3H), 0.92 (m, 4H), 1.13 (m, 2H), 1.28 (m, 2H), 1.31 and 1.32 (s, 9H), 1.98 (m, 1H), 4.69 (m, 1H), 7.15 (m, 1H), 7.42-7.48 (m, 2H), 8.52 (s, 1H). HPLC (Method B): retention time 7.18 min, m/z 449.1 [M+H]⁺.

### Example 32

### Synthesis of 4-(bicyclo[2.2.1]heptan-2-yloxy)-6-tert-butyl-5-(3,4-dichlorophenyl)thieno[2,3-d]pyrimidine (7ad)

Compound 7ad was prepared as described for the preparation of compound 7a with the exception that bicyclo[2.2.1]heptan-2-ol was used in place of 2-(trifluoromethoxy)phenol. The crude material was purified by Teledyne ISCO chromatography One using 0-20% EtOAc/hexanes as eluent to give 7ad as a white solid (12% yield). Mp 152° C.; ¹H NMR (CDCl₃): δ 0.41-0.49 (m, 1H), 0.89 (m, 2H), 1.12 (m, 1H), 1.28 (m, 2H), 1.31 and 1.30-1.46 (s, 12H), 1.85 (m, 1H), 2.12 (m, 1H), 2.46-2.51 (m, 1H), 5.21 (m, 1H), 7.17-7.19 (dt, J=2.5, 8.1 Hz, 1H), 7.44 (m, 1H), 7.47 (dd, J=2.5, 8.1 Hz, 1H), 8.53 (s, 1H). HPLC (Method B): retention time 6.97 min, m/z 447.1 [M+H]⁺.

### Example 33

### Synthesis of 6-tert-butyl-4-(cycloheptyloxy)-5-(3,4-dichlorophenyl)thieno[2,3-d]pyrimidine (7ae)

Compound 7ae was prepared as described for the preparation of compound 7a with the exception that cycloheptanol was used in place of 2-(trifluoromethoxy)phenol. The crude material was purified by preparative HPLC (Method D) to afford 7ae as a white solid (19% yield). ¹H NMR (CDCl₃): δ 1.26-1.31 (m, 13H), 1.46 (m, 4H), 1.62 (m, 2H), 1.76 (m, 2H), 5.31 (m, 1H), 7.15 (m, 1H), 7.42-7.46 (m, 2H), 8.52 (s, 1H). HPLC (Method B): retention time 7.29 min, m/z 449.2 [M+H]⁺.

### Example 34

### Synthesis of 6-tert-butyl-5-(3,4-dichlorophenyl)-4-(2-(trifluoromethyl)cyclohexyloxy)thieno[2,3-d]pyrimidine (7af)

Compound 7af was prepared as described for the preparation of compound 7a with the exception that 2-(trifluoromethyl)cyclohexanol was used in place of 2-(trifluoromethoxy)phenol. The crude material was purified by preparative HPLC (Method C) to afford a 1.5:1 mixture of diastereomer 7af as a white solid (21% yield). Mp 139° C.; ¹H NMR (CDCl₃): δ 1.16-1.22 (m, 2H), 1.27 (s, 9H), 1.37 (m, 2H), 1.46 (m, 1H), 1.55 (m, 1H), 1.66 (m, 1H), 1.8-1.97 (m, 1H), 2.08 (m, 1H), 6.01 (m, 1H), 7.16-7.23 (m, 1H), 7.46-7.51 (m, 2H), 8.55 (s, 1H). HPLC (Method A): retention time 3.25 and 3.39 min, m/z 503.1 [M+H]⁺.

### Example 35

### Synthesis of 6-tert-butyl-5-(3,4-dichlorophenyl)-4-(2-fluorocyclohexyloxy)thieno[2,3-d]pyrimidine (7ag)

Compound 7ag was prepared as described for the preparation of compound 7a with the exception that 2-fluorocyclohexanol was used in place of 2-(trifluoromethoxy)phenol. The crude material was triturated in a mixture of methanol and water to 7ag as a white solid (77% yield). Mp 144° C.; ¹H NMR (CDCl₃): δ 0.88-1.08 (m, 2H), 1.22-1.40 (s, 12H), 1.67 (m, 1H), 1.76 (m, 1H), 2.01 (m, 1H), 3.92 (m, 1H), 5.25 (m, 1H), 7.16-7.22 (m, 1H), 7.32-7.51 (m, 2H), 8.54 (s, 1H). HPLC (Method A): retention time 3.16 min, m/z 453.1 [M+H]⁺.

### Example 36

### Synthesis of 6-tert-butyl-5-(3,4-dichlorophenyl)-4-(2,2-dimethylcyclohexyloxy)thieno[2,3-d]pyrimidine (7ah)

Compound 7ah was prepared as described for the preparation of compound 7a with the exception that 2,2-dimethylcyclohexanol was used in place of 2-(trifluoromethoxy)phenol. The crude material was purified by preparative HPLC (Method D) to afford 7ah as a white solid (24% yield). Mp 128° C.; ¹H NMR (CDCl₃): δ 0.40 (d, J=13.1 Hz, 1H), 0.79 (d, J=11.3 Hz, 1H), 1.07 (m, 1H), 1.27-1.35 (s, 15H), 1.69 (m, 1H), 5.09 (m, 1H), 7.16-7.23 (m, 1H), 7.45-7.50 (m, 2H), 8.53 (s, 1H). HPLC (Method B): retention time 7.77 min, m/z 463.2 [M+H]⁺.

### Example 37

### Synthesis of 6-tert-butyl-5-(3,4-dichlorophenyl)-4-(1-(trifluoromethyl)cyclohexyloxy)thieno[2,3-d]pyrimidine (7a1)

Compound 7ai was prepared as described for the preparation of compound 7a with the exception that 1-(trifluoromethyl)cyclohexanol was used in place of 2-(trifluoromethoxy)phenol. The crude material was purified by preparative HPLC (Method A) to afford 7ai as a white solid (15% yield). Mp 142° C.; ¹H NMR (CDCl₃): δ 1.06-1.19 (m, 4H), 1.30 (s, 9H), 1.39-1.46 (m, 2H), 1.50-1.55 (m, 2H), 2.15 (m, 1H), 2.58 (m, 1H), 7.16 (dd, J=2.0, 8.1 Hz, 1H), 7.45-7.47 (m, 2H), 8.51 (s, 1H). HPLC (Method B): retention time 7.06 min, m/z 503.1 [M+H]⁺.

### Example 38

### Synthesis of 6-tert-butyl-5-(3,4-difluorophenyl)-4-(2-fluorophenoxy)thieno[2,3-d]pyrimidine (7aj)

### 6-tert-Butyl-5-(3,4-difluorophenyl)-4-methoxythieno[2, 3-d]pyrimidine (4b)

Compound 4b was prepared as described for the preparation of compound 4a with the exception that 3,4-difluorophenylboronic acid was used in place of 3,4-dichlorophenylboronic acid. (27% yield). ¹H NMR (CDCl₃) δ 1.29 (s, 9H), 3.72 (s, 3H), 6.98-7.20 (m, 3H), 8.57 (s, 1H).

### 6-tert-Butyl-5-(3,4-difluorophenyl)thieno[2, 3-d]pyrimidin-4-ol (5b)

Compound 5b was prepared as described for the preparation of compound 5a with the exception that the reaction was stirred overnight. (50% yield). ¹H NMR (CDCl₃): δ 1.27 (s, 9H), 7.03 (m, 1H), 7.11-7.19 (m, 2H), 7.84 (s, 1H), 9.83 (s, 1H).

### 6-tert-Butyl-4-chloro-5-(3,4-difluorophenyl)thieno[2,3-d]pyrimidine (6b)

Compound 6b was prepared as described for the preparation of compound 6a with the exception that the reaction was stirred overnight. (84% yield). ¹H NMR (CDCl₃): δ 1.32 (s, 9H), 7.04 (m, 1H), 7.15-7.24 (m, 2H), 8.78 (s, 1H).

### 6-tert-Butyl-5-(3,4-difluorophenyl)-4-(2-fluorophenoxy)thieno[2,3-d]pyrimidine (7aj)

Compound 7aj was prepared as described for the preparation of compound 7a with the exception that compound 6b was used in place of compound 6a and 4-fluorophenol was used in place of 2-trifluoromethoxyphenol. The residual material was purified by Biotage Isolera One using EtOAc/hexanes as eluent, followed by recrystallization in EtOAc to afford 7aj as a white solid (48% yield). Mp 171° C.; ¹H NMR (DMSO-d₆) δ 1.30 (s, 9H), 7.11-7.21 (m, 2H), 7.24-7.35 (m, 3H), 7.43-7.50 (m, 1H), 7.62-7.67 (m, 1H), 8.57 (s, 1H). HPLC (Method A): retention time 2.50 min, m/z 415.1 [M+H]⁺.

### Example 39

### Synthesis of 6-tert-butyl-4-(2,6-difluorophenoxy)-5-(3,4-difluorophenyl)thieno[2,3-d]pyrimidine (7ak)

Compound 7ak was prepared as described for the preparation of compound 7a with the exception that compound 6b was used in place of compound 6a and 2,6 difluorophenol was used in place of 2-trifluoromethoxyphenol. The residual material was purified by Biotage Isolera One using EtOAc/hexanes as eluent, followed by recrystallization in EtOAc to afford 7ak as a white solid (66% yield). Mp 185° C.; ¹H NMR (DMSO-d₆) δ 1.31 (s, 9H), 7.20-7.25 (m, 2H), 7.30-7.38 (m, 2H), 7.44-7.51 (m, 1H), 7.60-7.66 (m, 1H), 8.61 (s, 1H). HPLC (Method A): retention time 2.54 min, m/z 433.1 [M+H]⁺.

### Example 40

### Synthesis of 6-tert-butyl-5-(3,4-difluorophenyl)-4-(2-fluoro-6-(trifluoromethoxy)phenoxy)thieno[2,3-d]pyrimidine (7a1)

Compound 7a1 was prepared as described for the preparation of compound 7a with the exception that compound 6b was used in place of compound 6a and 2-fluoro-6-(trifluoromethoxy)phenol was used in place of 2-trifluoromethoxyphenol. The residual material was purified by Biotage Isolera One using EtOAc/hexanes as eluent, followed by recrystallization in EtOAc to afford 7a1 as a white solid (8% yield). Mp 150° C.; ¹H NMR (DMSO-d₆) δ 1.31 (s, 9H), 7.24-7.65 (m, 6H), 8.60 (s, 1H). HPLC (Method A): retention time 2.68 min, m/z 499.1 [M+H]⁺.

### Example 41

Synthesis of 6-tert-butyl-5-(3,4-difluorophenyl)-4-(2-(trifluoromethoxy)phenoxy)thieno[2,3-d]pyrimidine (7am)

Compound 7am was prepared as described for the preparation of compound 7a with the exception that compound 6b was used in place of compound 6a. The residual material was purified by Biotage Isolera One using EtOAc/hexanes as eluent, followed by recrystallization in EtOAc to afford 7am as a white solid (14% yield). Mp 146° C.; ¹H NMR (DMSO-d₆) δ 1.30 (s, 9H), 7.24-7.47 (m, 6H), 7.53-7.58 (m, 1H), 8.55 (s, 1H). HPLC (Method A): retention time 2.58 min, m/z 481.1 [M+H]⁺.

### Example 42

### Synthesis of 6-tert-butyl-5-(4-chlorophenyl)-4-(2-(trifluoromethoxy)phenoxy)thieno[2,3-d]pyrimidine (7an)

### 6-tert-Butyl-5-(4-chlorophenyl)-4-methoxythieno[2, 3-d]pyrimidine (4c)

Compound 4c was prepared as described for the preparation of compound 4a with the exception that 4-chlorophenylboronic acid was used in place of 3,4-dichlorophenylboronic acid. (62% yield). ¹H NMR (CDCl₃) δ 1.28 (s, 9H), 3.70 (s, 3H), 7.20 (d, J=8.3 Hz, 2H), 7.35 (d, J=8.3 Hz, 2H), 8.57 (s, 1H).

### 6-tert-Butyl-5-(4-chlorophenyl)thieno[2,3-d]pyrimidin-4-ol (5c)

Compound 5c was prepared as described for the preparation of compound 5a with the exception that the crude material was used as is without further purification. White solid (quantitative yield). ¹H NMR (CDCl₃) δ 1.26 (s, 9H), 7.22 (d, J=8.6 Hz, 2H), 7.36 (d, J=8.3 Hz, 2H), 7.85 (s, 1H), 10.77 (bs, 1H).

### 6-tert-Butyl-4-chloro-5-(4-chlorophenyl)thieno[2,3-d]pyrimidine (6c)

Compound 6c was prepared as described for the preparation of compound 6a with the exception that the crude material was purified by Teledyne Isco using EtOAc/DCM as eluent. White solid (25% yield). ¹H NMR (CDCl₃) δ 1.31 (s, 9H), 7.25 (d, J=8.6 Hz, 2H), 7.41 (d, J=8.6 Hz, 2H), 8.78 (s, 1H). 6-tert-Butyl-5-(4-chlorophenyl)-4-(2-(trifluoromethoxy)phenoxy)thieno[2,3 d]pyrimidine (7an) Compound 7an was prepared as described for the preparation of compound 7a with the exception that compound 6c was used in place of compound 6a. The crude material was triturated in a mixture of methanol and water to afford 7an as a white solid (97% yield). Mp 136° C.; ¹H NMR (CDCl₃) δ 1.33 (s, 9H), 7.01 (m, 1H), 7.29-7.32 (m, 7H), 8.50 (s, 1H). HPLC (Method A): retention time 2.57 min, m/z 479.1 [M+H]⁺.

### Example 43

### Synthesis of 6-tert-butyl-5-(4-chlorophenyl)-4-(2-fluoro-6-(trifluoromethoxy)phenoxy)thieno[2,3-d]pyrimidine (7ao)

Compound 7ao was prepared as described for the preparation of compound 7a with the exception that compound 6c was used in place of compound 6a and 2-fluoro-6-(trifluoromethoxy)phenol was used in place of 2-trifluoromethoxyphenol. The crude product was purified by preparative HPLC (Method C) to afford 7ao as a white solid (38% yield). Mp 115° C.; ¹H NMR (CDCl₃): δ 1.33 (s, 9H), 7.10 (m, 2H), 7.21 (m, 1H), 7.33 (s, 4H), 8.49 (s, 1H). HPLC (Method A): retention time 2.99 min, m/z 497.1 [M+H]⁺.

### Example 44

### Synthesis of 6-tert-butyl-5-(4-chlorophenyl)-4-(2,6-difluorophenoxy)thieno[2,3-d]pyrimidine (7ap)

Compound 7ap was prepared as described for the preparation of compound 7a with the exception that compound 6c was used in place of compound 6a and 2,6 difluorophenol was used in place of 2-trifluoromethoxyphenol. The crude material was triturated in a mixture of methanol and water to afford 7ap as a white solid (67% yield). Mp 176° C.; ¹H NMR (CDCl₃): δ 1.33 (s, 9H), 6.94 (m, 2H), 7.14 (m, 1H), 7.36 (s, 4H), 8.51 (s, 1H). HPLC (Method A): retention time 2.79 min, m/z 431.1 [M+H]⁺.

### Example 45

### Synthesis of 6-tert-butyl-5-(3-chlorophenyl)-4-(2-(trifluoromethoxy)phenoxy)thieno[2,3-d]pyrimidine (7aq)

### 6-tert-Butyl-5-(3-chlorophenyl)-4-methoxythieno[2, 3-d]pyrimidine (4d)

Compound 4d was prepared as described for the preparation of compound 4a with the exception that 3-chlorophenylboronic acid was used in place of 3,4-dichlorophenylboronic acid. (80% yield). ¹H NMR (CDCl₃) δ 1.29 (s, 9H), 3.71 (s, 3H), 7.17 (m, 1H), 7.29-7.33 (m, 2H), 7.36 (m, 1H), 8.57 (s, 1H).

### 6-tert-Butyl-5-(3-chlorophenyl)thieno[2,3-d]pyrimidin-4-ol (5d)

Compound 5d was prepared as described for the preparation of compound 5a with the exception that the crude material was used as is without further purification. White solid (quantitative yield). ¹H NMR (CDCl₃) δ 1.27 (s, 9H), 7.18 (m, 1H), 7.30-7.33 (m, 2H), 7.37 (m, 1H), 7.77 (s, 1H), 10.43 (s, 1H).

### 6-tert-Butyl-4-chloro-5-(3-chlorophenyl)thieno[2,3-d]pyrimidine (6d)

Compound 6d was prepared as described for the preparation of compound 6a with the exception that the crude material was purified by Teledyne Isco using EtOAc/DCM as eluent. Off white solid (34% yield). ¹H NMR (CDCl₃) δ 1.32 (s, 9H), 7.21 (m, 1H), 734-7.39 (m, 2H), 7.44 (m, 1H), 8.78 (s, 1H).

### 6-tert-Butyl-5-(3-chlorophenyl)-4-(2-(trifluoromethoxy)phenoxy)thieno[2,3-d]pyrimidine (7aq)

Compound 7aq was prepared as described for the preparation of compound 7a with the exception that compound 6d was used in place of compound 6a. The crude material was triturated in a mixture of methanol and water to afford 7aq as a white solid (56% yield). Mp 129° C.; ¹H NMR (CDCl₃) δ 1.35 (s, 9H), 7.01 (m, 1H), 7.29-7.32 (m, 6H), 7.40 (s, 1H), 8.50 (s, 1H). HPLC (Method A): retention time 2.73 min, m/z 479.1 [M+H]⁺.

### Example 46

### Synthesis of 6-tert-butyl-5-(3-chlorophenyl)-4-(2-fluoro-6-(trifluoromethoxy)phenoxy)thieno[2,3-d]pyrimidine (7ar)

Compound 7ar was prepared as described for the preparation of compound 7a with the exception that compound 6d was used in place of compound 6a and 2,6-difluorophenol was used in place of 2-trifluoromethoxyphenol. The crude material was triturated in a mixture of methanol and water to afford 7ar as a tan solid (64% yield). Mp 126° C.; ¹H NMR (CDCl₃): δ 1.35 (s, 9H), 7.09 (m, 2H), 7.12 (m, 1H), 7.30 (s, 3H), 7.42 (m, 1H), 8.50 (s, 1H). HPLC (Method A): retention time 2.86 min, m/z 497.1 [M+H]⁺.

### Example 47

### Synthesis of 6-tert-butyl-5-(3-chlorophenyl)-4-(2,6-difluorophenoxy)thieno[2,3-d]pyrimidine (7as)

Compound 7as was prepared as described for the preparation of compound 7a with the exception that compound 6d was used in place of compound 6a and 2,6-difluorophenol was used in place of 2-trifluoromethoxyphenol. The crude material was triturated in a mixture of methanol and water to afford 7as as a white solid (99% yield). Mp 146° C.; ¹H NMR (CDCl₃): δ 1.34 (s, 9H), 6.94 (m, 2H), 7.14 (m, 1H), 7.32 (s, 3H), 7.45 (m, 1H), 8.52 (s, 1H). HPLC (Method A): retention time 2.57 min, m/z 431.1 [M+H]⁺.

### Example 48

### Synthesis of 6-tert-butyl-4-(2,6-difluorophenoxy)-5-(4-fluorophenyl)thieno[2,3-d]pyrimidine (7at)

### 6-tert-Butyl-5-(4 fluorophenyl)-4-methoxythieno[2, 3-d]pyrimidine (4e)

Compound 4e was prepared as described for the preparation of compound 4a with the exception that 4-fluorophenylboronic acid was used in place of 3,4-dichlorophenylboronic acid, (92% yield). ¹H NMR (CDCl₃) δ 1.28 (s, 9H), 3.70 (s, 3H), 7.07 (m, 2H), 7.23 (m, 2H), 8.57 (s, 1H).

### 6-tert-Butyl-5-(4 fluorophenyl)thieno[2, 3-d]pyrimidin-4-ol (5e)

Compound 5e was prepared as described for the preparation of compound 5a with the exception that the product was purified by Biotage Isolera One using EtOAc/hexanes containing 2% AcOH as eluent to give 5e as white solid (41% yield). ¹H NMR (CDCl₃) δ 1.26 (s, 9H), 7.09 (m, 2H), 7.24 (m, 2H), 7.78 (s, 1H), 10.58 (s, 1H).

### 6-tert-Butyl-4-chloro-5-(4 fluorophenyl)thieno[2, 3-d]pyrimidine (6e)

Compound 6e was prepared as described for the preparation of compound 6a with the exception that the crude material was purified by Biotage Isolera One using EtOAc/hexanes as eluent: (61% yield). ¹H NMR (CDCl₃) δ 1.31 (s, 9H), 7.13 (m, 2H), 7.28 (m, 2H), 7.44 (m, 1H), 8.78 (s, 1H).

### 6-tert-Butyl-4-(2,6-difluorophenoxy)-5-(4 fluorophenyl)thieno[2, 3-d]pyrimidine (7at)

Compound 7at was prepared as described for the preparation of compound 7a with the exception that compound 6e was used in place of compound 6a and 2,6-difluorophenol was used in place of 2-trifluoromethoxyphenol. The crude material was triturated in a mixture of methanol and water to afford 7at as a light yellow solid (73% yield). Mp 186° C.; ¹H NMR (CDCl₃): δ 1.34 (s, 9H), 6.94 (m, 2H), 7.14 (m, 1H), 7.32 (s, 3H), 7.45 (m, 1H), 8.52 (s, 1H). HPLC (Method A): retention time 2.73 min, m/z 415.2 [M+H]⁺.

### Example 49

### Synthesis of 6-tert-butyl-4-(2-fluorophenoxy)-5-(4-fluorophenyl)thieno[2,3-d]pyrimidine (7au)

Compound 7au was prepared as described for the preparation of compound 7a with the exception that compound 6e was used in place of compound 6a and 2-fluorophenol was used in place of 2-trifluoromethoxyphenol. The crude material was purified by Biotage Isolera One using EtOAc/hexanes as eluent to give 7au as a white solid (24% yield). Mp 194° C.; ¹H NMR (CDCl₃): δ 1.34 (s, 9H), 6.94 (m, 1H), 7.04-7.12 (m, 4H), 7.18 (m, 1H), 7.36 (m, 2H), 8.52 (s, 1H). HPLC (Method A): retention time 2.62 min, m/z 397.2 [M+H]⁺.

### Example 50

### Synthesis of 6-tert-butyl-5-(3-fluorophenyl)-4-(2-(trifluoromethoxy)phenoxy)thieno[2,3-d]pyrimidine (7av)

### 6-tert-Butyl-5-(3 fluorophenyl)-4-methoxythieno[2, 3-d]pyrimidine (4f)

Compound 4f was prepared as described for the preparation of compound 4a with the exception that 3-fluorophenylboronic acid was used in place of 3,4-dichlorophenylboronic acid, (69% yield). ¹H NMR (CDCl₃) δ 1.29 (s, 9H), 3.70 (s, 3H), 7.00 (m, 1H), 7.07 (m, 2H), 7.33 (m, 1H), 8.57 (s, 1H).

### 6-tert-Butyl-5-(3 fluorophenyl)thieno[2, 3-d]pyrimidin-4-ol (5f)

Compound 5f was prepared as described for the preparation of compound 5a with the exception that the material was used as is without further purification. White solid (quantitative yield). ¹H NMR (CDCl₃) δ 1.27 (s, 9H), 7.01 (m, 1H), 7.08-7.13 (m, 2H), 7.35 (m, 1H), 7.71 (s, 1H), 10.86 (s, 1H).

### 6-tert-Butyl-4-chloro-5-(3 fluorophenyl)thieno[2, 3-d]pyrimidine (6f)

Compound 6f was prepared as described for the preparation of compound 6a with the exception that the crude material was purified by Teledyne ISCO using EtOAc/hexanes as eluent: (67% yield). ¹H NMR (CDCl₃) δ 1.32 (s, 9H), 7.05 (m, 1H), 7.13 (m, 2H), 7.40 (m, 1H), 8.78 (s, 1H).

### 6-tert-Butyl-5-(3 fluorophenyl)-4-(2-(trifluoromethoxy)phenoxy)thieno[2,3-d]pyrimidine (7av)

Compound 7av was prepared as described for the preparation of compound 7a with the exception that compound 6f was used in place of compound 6a. The residual material was purified by preparative HPLC (Method B) to afford 7av as a white solid (41% yield). Mp 150° C.; ¹H NMR (CDCl₃): δ 1.35 (s, 9H), 7.03 (m, 2H), 7.09 (m, 1H), 7.16 (m, 1H), 7.26 (m, 1H), 7.28-7.31 (m, 3H), 8.50 (s, 1H). HPLC (Method A): retention time 2.66 min, m/z 463.1 [M+H]⁺.

### Example 51

### Synthesis of 6-tert-butyl-4-(2,6-difluorophenoxy)-5-(3-fluorophenyl)thieno[2,3-d]pyrimidine (7aw)

Compound 7aw was prepared as described for the preparation of compound 7a with the exception that compound 6f was used in place of compound 6a and 2,6 difluorophenol was used in place of 2-trifluoromethoxyphenol. The residual material was purified by preparative HPLC (Method B) to afford 7aw as a white solid (39% yield). Mp 180° C.; ¹H NMR (CDCl₃): δ 1.35 (s, 9H), 6.93 (m, 2H), 7.06 (m, 1H), 7.14 (m, 2H), 7.20 (m, 1H), 7.28-7.31 (m, 2H), 8.52 (s, 1H). HPLC (Method A): retention time 2.68 min, m/z 415.2 [M+H]+.

### Example 52

### Synthesis of 6-tert-butyl-5-(3,4-dichlorophenyl)-4-(2-(trifluoromethoxy)phenylthio)thieno[2,3-d]pyrimidine (7ax)

Compound 7ax was prepared as described for the preparation of compound 7a with the exception that 2-(trifluoromethoxy)benzenethiol was used in place of 2-(trifluoromethoxy)phenol. The crude material was triturated in a mixture of methanol and water to afford 7ax as a white solid (58% yield). Mp 165° C.; ¹H NMR (CDCl₃) δ 1.34 (s, 9H), 7.30-7.36 (m, 4H), 7.49 (m, 1H), 7.55 (m, 2H), 8.53 (s, 1H). HPLC (Method A) for 7ax: retention time 3.25 min, m/z 529.1 [M+H]⁺.

### Example 53

### Synthesis of 2-methyl-4-phenoxythieno[2,3-d]pyrimidine (7ay)

Compound 7ay was prepared as descried for the preparation of compound 7a with the exception that phenol was used in place of 2-(trifluoromethoxy)phenol and 4 chloro-2-methylthieno[2,3-d]pyrimidine (1b) in place of 6-tert-butyl-4-chloro-5-(3,4-dichlorophenyl)thieno[2,3-d]pyrimidine (6a). The crude material was purified by Biotage Isolera One using EtOAc/hexanes as eluent and the obtained yellow oil was triturated in methanol to give 7ay as a white solid (15% yield). Mp 100° C.; ¹H NMR (DMSO-d₆) δ 2.50 (s, 3H), 7.31 (m, 3H), 7.49 (m, 2H), 7.57 (d, J=6.1 Hz, 1H), 6.84 (d, J=5.8 Hz, 1H). HPLC (Method A): retention time 2.00 min, m/z 243.1 [M+H]⁺.

### Example 54

### Synthesis of 5,6-dimethyl-4-(o-tolyloxy)thieno[2,3-d]pyrimidine (7az)

Compound 7az was prepared as descried for the preparation of compound 7a with the exception that 2-methylphenol was used in place of 2-(trifluoromethoxy)phenol and 4-chloro-5,6-dimethylthieno[2,3-d]pyrimidine (1c) in place of 6-tert-butyl-4-chloro-5-(3,4-dichlorophenyl)thieno[2,3-d]pyrimidine (6a). The crude material was triturated in 10-20% EtOAc/hexanes to give 7az as a yellow solid (59% yield). Mp 107° C.; ¹H NMR (DMSO-d₆) δ 2.10 (s, 3H), 2.51 (s, 3H), 2.54 (s, 3H), 7.22 (m, 2H), 7.30 (m, 1H), 7.34 (m, 1H), 8.45 (s, 1H). HPLC (Method A): retention time 2.28 min, m/z 271.2 [M+H]⁺.

### Example 55

### Synthesis of 4-(2-fluorophenoxy)-5,6-dimethylthieno[2,3-d]pyrimidine (7ba)

Compound 7ba was prepared as descried for the preparation of compound 7a with the exception that 2-fluorolphenol was used in place of 2-(trifluoromethoxy)phenol and 4-chloro-5,6-dimethylthieno[2,3-d]pyrimidine (1c) in place of 6-tert-butyl-4-chloro-5-(3,4-dichlorophenyl)thieno[2,3-d]pyrimidine (6a). The crude material was purified by Biotage Isolera One using EtOAc/hexanes as eluent to give 7ba as a white solid (75% yield). Mp 144° C.; ¹H NMR (DMSO-d₆) δ 2.52 (s, 6H), 7.29 (m, 1H), 7.34 (m, 1H), 7.40 (m, 1H), 7.47 (m, 1H), 8.50 (s, 1H). HPLC (Method A): retention time 2.28 min, m/z 275.1 [M+H]⁺.

### Example 56

### Synthesis of 4-(2-fluorophenoxy)-6-methyl-5-phenylthieno[2,3-d]pyrimidine (7bb)

Compound 7bb was prepared as descried for the preparation of compound 7a with the exception that 2-fluorolphenol was used in place of 2 (trifluoromethoxy)phenol and 4-chloro-6-methyl-5-phenylthieno[2,3-d]pyrimidine (1d) in place of 6-tert-butyl-4-chloro-5-(3,4-dichlorophenyl)thieno[2,3-d]pyrimidine (6a). The crude material was triturated in a mixture of 20% EtOAc/hexanes and water to afford 7bb as a beige solid (38% yield). Mp 158° C.; ¹H NMR (CDCl₃) δ 2.49 (s, 3H), 7.07-7.21 (m, 4H), 7.35-7.43 (m, 5H), 8.54 (s, 1H). HPLC (Method A): retention time 3.42 min, m/z 337.1 [M+H]⁺.

### Example 57

### Synthesis of 6-(tert-butyl)-4-(2-fluorophenoxy)thieno[2,3-d]pyrimidine (7bc)

Compound 7bc was prepared as descried for the preparation of compound 7a with the exception that 2-fluorolphenol was used in place of 2-(trifluoromethoxy)phenol and 6-(tert-butyl)-4-chlorothieno[2,3-d]pyrimidine (1a) in place of 6-tert-butyl-4-chloro-5-(3,4-dichlorophenyl)thieno[2,3-d]pyrimidine (6a). The crude material was purified by Biotage Isolera One using EtOAc/hexanes as eluent to give 7bc as a colorless semi-solid (60% yield). ¹H NMR (CDCl₃) δ 1.52 (s, 9H), 7.25 (m, 1H), 7.28 (s, 1H), 7.29-7.34 (m, 3H), 8.55 (s, 1H). HPLC (Method A): retention time 2.53 min, m/z 303.1 [M+H]⁺.

### Example 58

### Synthesis of 6-(tert-butyl)-4-(2-fluorophenoxy)-5-phenylthieno[2,3-d]pyrimidine

### 7bd

### 6-tert-Butyl-5 phenylthieno[2,3-d]pyrimidin-4-ol (9)

In a vial, a mixture of 3,3-dimethyl-1-phenylbutan-1-one 8 (500 mg, 2.84 mmol), ethylcyanoacetate (483 mg, 4.26 mmol), sulfur (726 mg, 2.84 mmol), formamide (0.9 mL, 22.7 mmol), L-proline (65.3 mg, 0.57 mmol), and diethylamine (41.5 mg, 0.57 mmol) was stirred at 170° C. for 6 h. After the vial was cooled to rt, the residue was triturated with DCM. The resulting solid was removed by filtration and the filtrate was concentrated and purified by Biotage Isolera One using 30-50% EtOAc in DCM as eluent to give 35 mg (4%) of 9 as yellow solid. ¹H NMR (CDCl₃) δ 1.24 (s, 9H), 7.31-7.25 (m, 2H), 7.46-7.34 (m, 3H), 10.91 (s, 1H). HPLC (Method A): retention time 1.98 min, m/z 285.2 [M+H]⁺.

### 6-tert-Butyl-4-chloro-5phenylthieno[2,3-d]pyrimidine (10)

A solution of 9 (35 mg, 0.12 mmol) in POCl₃ (0.5 mL) was stirred at 110° C. for 1 h. Excess POCl₃ was removed in vacuo followed by co-evaporation with DCM three times. The crude was purified by Biotage Isolera One using 0-10% EtOAc in DCM as eluent to give 25 mg (66%) of 10 as a yellow oil. ¹H NMR (CDCl₃) δ 1.30 (s, 9H), 7.32-7.28 (m, 2H), 7.47-7.38 (m, 3H), 8.76 (s, 1H). HPLC (Method A): retention time 2.44 min, m/z 303.1 [M+H]⁺.

### 6-(tert-butyl)-4-(2 fluorophenoxy)-5 phenylthieno[2,3-d]pyrimidine (7bd)

Sodium hydride (60% dispersion in mineral oil, 5.3 mg, 0.13 mmol) was added to a solution of 2-fluorophenol (14.8 mg, 0.13 mmol) in DMF (0.2 mL) at 0° C. and the mixture was stirred at 0° C. for 5-10 min. A solution of 10 (20 mg, 0.07 mmol) in DMF (0.3 mL) was then added slowly to the mixture via syringe at 0° C. The reaction mixture was stirred and allowed to warm to rt overnight. A few drops of 10% KHSO₄ were added to the mixture to quench the excess NaH and the mixture was then concentrated in vacuo. The crude residue was triturated in methanol and water to give 10 mg (40%) of 7bd as a light yellow solid. Mp 81-82° C. ¹H NMR (CDCl₃) δ 1.33 (s, 9H), 6.92 (dt, J=8.4, 4.1 Hz, 1H), 7.17-7.03 (m, 3H), 7.44-7.27 (m, 5H), 8.50 (s, 1H). HPLC (Method A): retention time 2.64 min, m/z 379.2 [M+H]⁺.

### Example 59

### Synthesis of Ethyl 6-(tert-butyl)-4-(2-fluoro-6-(trifluoromethoxy)phenoxy)thieno[2,3 d]pyrimidine-5-carboxylate (7be)

### 6-(tert-Butyl)-4-(2 fluoro-6-(trifluoromethoxy)phenoxy)thieno[2,3-d]pyrimidine (11)

Sodium hydride (60% dispersion in mineral oil, 211 mg, 5.29 mmol) was added to a solution of 2-fluoro-6-(trifluoromethoxy)phenol (1.03 g, 5.29 mmol) in DMF (10 mL) at 0° C. and the mixture was stirred at 0° C. for 5-10 min. A solution of 1a (600 mg, 2.65 mmol) in DMF (5 mL) was then added slowly to the mixture via syringe at 0° C. The reaction mixture was stirred and allowed to warm to rt and heated to 60° C. overnight. A few drops of water were added to the mixture to quench the excess NaH and the mixture was then concentrated in vacuo. The crude residue was purified by Biotage Isolera One using 10-20% EtOAc in hexanes as eluent to give 960 mg of desired compound 11 as a white solid (94% yield); Mp 74-75° C. ¹H NMR (CDCl₃) δ 1.51 (s, 9H), 7.16-7.37 (m, 4H), 8.51 (s, 1H); HPLC (Method A): retention time 2.64 min, m/z 387.1 [M+H]⁺.

### 5-Bromo-6-(tert-butyl)-4-(2 fluoro-6-(trifluoromethoxy)phenoxy)thieno[2,3-d]pyrimidine (12)

To a solution of 11 (1.56 g, 7.02 mmol) in AcOH (30 mL) was added N-bromosuccinimide (3.75 g, 21.1 mmol) and the mixture was stirred at 55° C. overnight. The reaction mixture was concentrated, and the crude material was purified by Biotage Isolera One using 0-10% EtOAc in hexanes as eluent to give 1.8 g of the desired compound 12 as a yellow orange solid (85% yield). ¹H NMR (CDCl₃) δ 1.64 (s, 9H), 7.18-7.41 (m, 3H), 8.50 (s, 1H).

### Ethyl 6-(tert-butyl)-4-(2 fluoro-6-(trifluoromethoxy)phenoxy)thieno[2,3-d]pyrimidine-5-carboxylate (7be)

A solution of 12 (50.0 mg, 0.11 mmol) in THE was cooled to −78° C. nBuLi 2.5 M in hexanes (0.047 mL, 0.12 mmol) was added and the mixture was stirred for 30 min. Ethyl chloroformate (17.3 mg, 0.16 mmol) was then added slowly. The reaction mixture was stirred and allowed to warm to rt. The reaction was quenched with water and concentrated in vacuo. The crude material was purified by Biotage Isolera One using 10-30% EtOAc in hexanes as eluent to give 35 mg of desired compound 7be as a light yellow oil (70% yield); ¹H NMR (CDCl₃) δ 1.34 (t, J=7.2 Hz, 3H), 1.53 (s, 9H), 4.21-4.54 (m, 2H), 7.13-7.38 (m, 3H), 8.52 (s, 1H); HPLC (Method A): retention time 2.48 min, m/z 458.2 [M+H]⁺.

### Example 60

### Synthesis of 6-(tert-butyl)-4-(2-fluoro-6-(trifluoromethoxy)phenoxy)-5-(pyrrolidin-1-yl)thieno[2,3-d]pyrimidine (7b1)

### 6-(tert-Butyl)-5-(pyrrolidin-1 yl)thieno[2,3-d]pyrimidin-4-ol (13)

To a sealed tube containing compound 3 (0.30 g, 1.00 mmol) was added pyrrolidine (2.4 mL) and 4.8 mL of water. A small spatula tip amount of copper (I) oxide was added, and the reaction was heated at 120° C. for 5 h. The mixture was transferred to a round bottom flask and concentrated in vacuo. The crude material was partitioned between 10% KHSO₄ an ethyl acetate. The organic layer was washed with brine, dried over sodium sulfate, and concentrated. The residual solid was triturated in a mixture of methanol, dichloromethane, and ethyl acetate to give 65 mg (24% yield) of the desired product 13 as a white solid. ¹H NMR (CDCl₃) δ 1.47 (s, 9H), 1.94 (s, 3H), 2.07 (s, 2H), 3.06 (s, 2H), 3.20 (s, 2H), 7.89 (s, 1H), 10.44 (s, 1H)

### 6-(tert-Butyl)-4-chloro-5-(pyrrolidin-1 yl)thieno[2,3-d]pyrimidine (14)

A solution of 13 (65 mg, 0.23 mmol) in POCl₃ (3 mL) was stirred at 110° C. for 2 days. Excess POCl₃ was removed in vacuo followed by co-evaporation with DCM (three times). The residual material was passed through a short silica column using 10-45% EtOAc in hexanes as eluent to give 35 mg (51%) of the desired product as a yellow semi solid. ¹H NMR (CDCl₃) δ 1.51 (s, 9H), 1.99 (s, 2H), 2.09 (s, 2H), 3.20 (s, 2H), 3.42 (s, 2H), 8.75 (s, 1H).

### 6-(tert-butyl)-4-(2 fluoro-6-(trifluoromethoxy)phenoxy)-5-(pyrrolidin-1 yl)thieno[2,3-d]pyrimidine (7bf)

To a solution of 2-fluoro-6-(trifluoromethoxy)phenol (35 mg, 0.18 mmol) in DMF (2 mL) was added sodium hydride (60% dispersion in mineral oil, 7.0 mg, 0.18 mmol) at rt and the mixture was stirred for 5-10 min. A solution of 4 (35 mg, 0.12 mmol) in DMF (1 mL) was added to the mixture via syringe. The reaction was heated at 60° C. over weekend. A few drops of 10% KHSO₄ was added to quench excess of NaH and the mixture was concentrated in vaccuo. The residual material was purified by prep-HPLC (Method C) to give 32 mg (59%) of 7bf as a beige solid. Mp 115° C. ¹H NMR (CDCl₃) δ 1.54 (s, 9H), 1.92 (s, 4H), 3.19 (s, 2H), 3.36 (s, 2H), 7.21 (s, 1H), 7.34 (s, 2H), 8.47 (s, 1H). HPLC (method A): >99% by area, retention time 3.04 min, m/z 456.2 [M+H]⁺.

### Example 61

### Synthesis of 6-(tert-butyl)-4-(2-fluoro-6-(trifluoromethoxy)phenoxy)-5-(2-methylpyrrolidin-1-yl)thieno[2,3-d]pyrimidine (7bg)

### 6-(tert-butyl)-5-nitrothieno[2,3-d]pyrimidin-4-ol (15)

6-(tert-Butyl)thieno[2,3-d]pyrimidin-4-ol (2.0 g, 9.60 mmol) was added as a solid to a 1:1 mixture of nitric acid and conc. sulfuric acid (20 mL) at 0° C. The resulting mixture was stirred at rt overnight. Additional amount of nitric acid was added (0.5 mL) and stirring continued for another 3h. Ice water (33 mL) was then slowly added and the resulting precipitate was filtered, washed thoroughly with cold water and dried under in-house vacuum to give 2.18 g (90% yield) of 15 as a yellow solid. ¹H NMR (DMSO) δ 1.39 (s, 9H), 8.23 (s, 1H), 12.93 (s, 1H).

### 5Amino-6-(tert-butyl)thieno[2,3-d]pyrimidin-4-ol (16)

To a suspension of 15 (2.18 g, 8.61 mmol) in 1:1 MeOH/DCM (84 mL) was added Zn dust (5.63 g, 81.6 mmol), followed by a dropwise addition of saturated NH₄Cl solution (42 mL) at rt. After 1 h stirring, the reaction was filtered through celite, washed with a mixture of MeOH/DCM. The layers were separated. The organic layer was dried over sodium sulfate and concentrated in vacuo to give the first crop of the desired product. The mother liquor was concentrated, and the second crop of the product was obtained by precipitation from a mixture of DCM/water. A total of 1.67 g (87% yield) of compound 16 was obtained as a yellow solid. ¹H NMR (DMSO) δ 1.36 (s, 9H), 5.23 (s, 2H), 7.95 (s, 1H), 12.27 (s, 1H).

### 6-(tert-Butyl)-5-(2-methylpyrrolidin-1 yl)thieno[2,3-d]pyrimidin-4-ol (17a)

To a solution of oxopentanal (0.25 g, 2.50 mmol) and 16 (0.20 g, 0.90 mmol) in methanol (15 mL) was added a spec of bromocresol green followed by sodium cyanoborohydride (0.23 g, 3.58 mmol) in one portion at rt. Acetic acid was then added dropwise until the color of the mixture turned from bluish to clear green and then yellow. The reaction mixture was stirred overnight and the solvent was removed in vacuo. The crude material was partitioned between water and dichloromethane. The organic layer was washed with brine, dried over sodium sulfate, and concentrated. The residue was purified by Biotage Isolera One using EtOAc in hexanes in the presence of 2% AcOH as eluent) to give 0.12 g (44% yield) of the desired product 17a as a yellow solid foam. 1H NMR (CDCl3) δ 0.97 (d, J=6.3 Hz, 3H), 1.47 (m, 1H), 1.51 (s, 9H), 1.87 (m, 1H), 2.01 (m, 1H), 2.20 (m, 1H), 3.17 (m, 2H), 3.69 (m, 1H), 7.91 (s, 1H), 11.18 (s, 1H).

### 6-(tert-butyl)-4-chloro-5-(2-methylpyrrolidin-1 yl)thieno[2, 3-d]pyrimidine (18a)

A solution of 17a (0.11 mg, 0.38 mmol) in POCl₃ (3 mL) was stirred at 110° C. for 24 h. Excess POCl₃ was removed in vacuo followed by co-evaporation with DCM (three times). The residual material was passed through a short silica column using 10-45% EtOAc in hexanes as eluent to give 60 mg (51%) of the desired product 18a as a yellow semi-solid.

### 6-(tert-Butyl)-4-(2 fluoro-6-(trifluoromethoxy)phenoxy)-5-(2-methylpyrrolidin-1-yl)thieno[2, 3-d]pyrimidine (7bg)

To a solution of 2-fluoro-6-(trifluoromethoxy)phenol (20 mg, 0.10 mmol) in DMF (2 mL) was added sodium hydride (60% dispersion in mineral oil, 4 mg, 0.10 mmol) at rt and the mixture was stirred 5-10 min. A solution of 6-(tert-butyl)-4-chloro-5-(2-methylpyrrolidin-1-yl)thieno[2,3-d]pyrimidine (20 mg, 0.06 mmol) in DMF (1 mL) was added to the mixture via syringe. The reaction mixture was heated at 60° C. for 48 h. A few drops of 10% KHSO₄ was added to the mixture to quench excess of NaH and the mixture was concentrated in vaccuo. The residual material was purified by prep-HPLC (Method C) to give 15 mg (50%) of 7bg as a brown solid. Mp 113° C. 1H NMR (CDCl₃) δ 1.05 (d, J=6.2 Hz, 3H), 1.52 (m, 1H), 1.56 (s, 9H), 1.87 (m, 2H), 2.06 (m, 1H), 3.21-3.44 (m, 2H), 3.69-3.94 (m, 2H), 7.22 (m, 2H), 7.33 (m, 1H), 8.46 (s, 1H).

### Example 62

### Synthesis of 6-(tert-butyl)-4-(2-fluoro-6-(trifluoromethoxy)phenoxy)-5-(piperidin-1-yl)thieno[2,3-d]pyrimidine (7bh)

### 6-(tert-Butyl)-5-(piperidin-1 yl)thieno[2, 3-d]pyrimidin-4-ol (17b)

Compound 17b was prepared from 16 as described for the preparation of compound 17a with the exception that glutaraldehyde was used in place of oxopentanal (75% yield). ¹H NMR (DMSO) δ 1.22-1.37 (m, 1H), 1.44 (s, 9H), 1.49-1.67 (m, 4H), 1.74 (d, J=13.0 Hz, 1H), 2.55 (d, J=10.1 Hz, 2H), 3.42 (td, J=11.2, 3.2 Hz, 2H), 8.04 (s, 1H), 12.32 (s, 1H).

### 6-(tert-Butyl)-4-chloro-5-(piperidin-1 yl)thieno[2,3-d]pyrimidine (18b)

Compound 18b was prepared from 17b as described for the preparation of compound 18b. (19% yield). 1H NMR (CDCl₃) δ 1.41 (m, 1H), 1.57 (s, 9H), 1.64-1.82 (m, 4H), 1.86 (d, J=13.1 Hz, 1H), 2.78 (d, J=9.6 Hz, 2H), 3.63 (td, J=11.2, 4.2 Hz, 2H), 8.76 (s, 1H).

### 6-(tert-Butyl)-4-(2 fluoro-6-(trifluoromethoxy)phenoxy)-5-(piperidin-1 yl)thieno[2,3-d]pyrimidine (7bh)

Compound 7bh was prepared from 18b as described for the preparation of 7bg. White solid (49% yield). Mp 133° C.; 1H NMR (400 MHz, CDCl₃) δ 1.25 (m, 1H), 1.56 (s, 4H), 1.58 (s, 9H), 1.63-1.88 (m, 5H), 2.84 (m, 2H), 3.30 (m, 1H), 3.46 (m, 1H), 7.22 (m, 1H), 7.33 (m, 2H), 8.47 (s, 1H). HPLC (method A): retention time 3.52 min, m/z 470.2 [M+H]⁺.

### Example 63

### Synthesis of 4-(6-(tert-butyl)-4-(2,6-difluorophenoxy)thieno[2,3-d]pyrimidin-5-yl)morpholine (7bi)

### 6-(tert-Butyl)-5-morpholinothieno[2, 3-d]pyrimidin-4-ol (17c)

Compound 17c was prepared from 16 as described for the preparation of compound 17a with the exception that 2,2′-oxydiacetaldehyde was used in place of oxopentanal (53% yield). ¹H NMR (DMSO) δ 1.46 (s, 9H), 2.42 (d, J=10.6 Hz, 2H), 3.51-3.74 (m, 4H), 3.80 (d, J=9.3 Hz, 2H), 8.07 (s, 1H), 12.44 (s, 1H).

### 4-(6-(tert-Butyl)-4-chlorothieno[2, 3-d]pyrimidin-5 yl)morpholine (18c)

Compound 18c was prepared from 17c as described for the preparation of compound 18a. White solid (88% yield). ¹H NMR (CDCl₃) δ 1.57 (s, 9H), 2.66 (m, 2H), 3.78-3.92 (m, 2H), 3.91-4.11 (m, 4H), 8.78 (s, 1H).

### 4-(6-(tert-Butyl)-4-(2,6-difluorophenoxy)thieno[2,3-d]pyrimidin-5 yl)morpholine (7bi)

Compound 7bi was prepared from 18c as described for the preparation of 7bg with the exception that 2,6-difluorophenol was used in place of 2-fluoro-6-(trifluoromethoxy)phenol and that the crude material was triturated in a mixture of methanol and water to afford 7bi as an off-white solid (96% yield). Mp 173° C. ¹H NMR (CDCl₃) δ 1.59 (s, 9H), 2.70 (m, 2H), 3.62-4.12 (m, 6H), 7.08 (m, 2H), 7.31 (m, 1H), 8.52 (s, 1H). HPLC (method A): retention time 2.54 min, m/z 406.2 [M+H]⁺.

### Example 64

### Synthesis of 6-(tert-butyl)-N,N-diethyl-4-(2-fluoro-6-(trifluoromethoxy)phenoxy)thieno[2,3-d]pyrimidin-5-amine (7bj)

### 6-(tert-Butyl)-5-(diethylamino)thieno[2,3 d]pyrimidin 4 of (17d)

Compound 17d was prepared from 16 as described for the preparation of compound 18a with the exception that acetaldehyde was used in place of oxopentanal and the crude material was carried to the next step without further purification. Yellow oil (66% yield). ¹H NMR (CDCl₃) δ 1.06 (t, J=7.3 Hz, 6H), 1.52 (s, 9H), 2.95 (qt, J=12.3, 7.5 Hz, 2H), 3.25 (qt, J=12.3, 7.2 Hz, 2H), 7.93 (s, 1H), 10.90 (s, 1H).

### 6-(tert-Butyl)-4-chloro-N,N-diethylthieno[2,3-d]pyrimidin-5-amine (18d)

Compound 18d was prepared from 17d as described for the preparation of compound 18a with the exception that one equivalent of triethylamine was added to the reaction. Yellow solid (34% yield). ¹H NMR (400 MHz, CDCl₃) δ 1.04 (t, J=7.4 Hz, 6H), 1.55 (s, 9H), 3.01 (qt, J=11.9, 7.5 Hz, 2H), 3.35 (qt, J=11.9, 7.2 Hz, 2H), 8.75 (s, 1H).

### 6-(tert-Butyl)-N,N-diethyl-4-(2 fluoro-6-(trifluoromethoxy)phenoxy)thieno[2,3-d]pyrimidin-5-amine (7bj)

Compound 7bj was prepared as described for the preparation of 7bg. White solid (67% yield). Mp 72° C.; ¹H NMR (CDCl₃) δ 1.09 (td, J=7.3, 5.3 Hz, 6H), 1.58 (s, 9H), 2.92-3.12 (m, 2H), 3.18-3.38 (m, 2H), 7.17-7.25 (m, 1H), 7.32 (m, 1H), 8.46 (s, 1H). HPLC (method A): retention time 3.28 min, m/z 458.3 [M+H]⁺.

### Example 65

### Synthesis of (6-(tert-butyl)-4-(2-fluoro-6-(trifluoromethoxy)phenoxy)furo[2,3-d]pyrimidin-5-yl)(piperidin-1-yl)methanone (7bk)

### Ethyl 2-bromo-4,4-dimethyl-3-oxopentanoate (19)

N-Bromosuccinimide (1.08 g, 6.10 mmol) was added to a solution of ethyl 4,4-dimethyl-3-oxopentanoate (1.00 g, 5.81 mmol) in DMSO (10 mL). The mixture was stirred at rt for 1 h. The reaction was quenched with water and extracted with EtOAc (3×20 mL). The organic layers were combined, dried over sodium sulfate, and concentrated in vacuo. The crude yellow oil 19 (1.46 g) was used in next step without purification. HPLC (Method A): retention time 1.88 min, m/z 251.1 [M+H]⁺.

### Ethyl 5-amino-2-(tert-butyl)-4-cyanofuran-3-carboxylate (20)

To a solution of 19 (1.46 g) in EtOH (40 mL) was added malononitrile (384 mg, 5.81 mmol) and sodium ethoxide (791 mg, 11.6 mmol). The reaction was stirred at rt overnight. Water was added to the reaction mixture and extracted with EtOAc. The organic layers were combined and dried over sodium sulfate and concentrated in vacuo. The crude material was purified by Biotage Isolera One using 0-10% methanol in dichloromethane as eluent to give 701 mg of the desired compound 20 as a dark brown oil (51% yield over two steps).

### Ethyl 6-(tert-butyl)-4-hydroxyfuro[2,3-d]pyrimidine-5-carboxylate (21)

A solution of 20 (701 mg, 2.97 mmol) in formic acid (5 mL) was cooled to 0° C. Acetic anhydride (5 mL) was added slowly the mixture was stirred at 0° C. for 1 h.

The reaction mixture was then heated to 100° C. for 24 h. The reaction was quenched with ice water and extracted with EtOAc (3×30 mL). The combined organic layers were dried over sodium sulfate and concentrated in vacuo. The crude material was purified by Biotage Isolera One using 0-10% methanol in dichloromethane as eluent to give 382 mg of the desired compound 21 as a brown solid (49% yield).

### 6-(tert-Butyl)-4-hydroxyfuro[2,3-d]pyrimidine-5-carboxylic acid (22)

Compound 21 (1.45 g, 5.49 mmol) was added to a solution of 6 M HCl (5 mL). The reaction was stirred and heated at 100° C. for 6 h. After cooling the reaction to 0° C., the white precipitate was collected to give 1.10 g of desired compound 22 as a white solid (85% yield). ¹H NMR (CDCl₃) δ 1.58 (s, 9H), 8.17 (s, 1H), 11.23 (s, 1H), 13.70 (s, 1H).

### (6-(tert-Butyl)-4-hydroxyfuro[2,3-d]pyrimidin-5 yl)(piperidin-1 yl)methanone (23)

To a solution of 22 (200 mg, 0.85 mmol) in DMF (1 mL) was added piperidine (66.2 mg, 0.93 mmol), HATU (354 mg, 0.93 mmol), and DIEA (0.47 mL, 3.39 mmol). The reaction was stirred at rt overnight. The reaction was concentrated, and the crude material was purified by Biotage Isolera One using 0-10% methanol in dichloromethane as eluent to give 108 mg of the desired compound 23 as a white solid (42% yield).

### (6-(tert-Butyl)-4-chlorofuro[2,3-d]pyrimidin-5 yl)(piperidin-1 yl)methanone (24)

Compound 23 (108 mg, 0.36 mmol) was dissolved in POCl₃ (1.04 mL, 11.2 mmol). The reaction was stirred at 110° C. for 1 h. The reaction mixture was diluted with CH₂Cl₂ and concentrated in vacuo. The crude material was purified by Biotage Isolera One using 10-30% EtOAc in dichloromethane as eluent to give 63 mg of the desired compound 24 as a white solid (55% yield). ¹H NMR (CDCl₃) δ 1.44 (s, 9H), 1.51-1.83 (m, 6H), 3.30 (td, J=7.8, 4.1 Hz, 2H), 3.78 (t, J=5.5 Hz, 2H), 8.73 (s, 1H).

### (6-(tert-Butyl)-4-(2 fluoro-6-(trifluoromethoxy)phenoxy)furo[2,3-d]pyrimidin-5-yl)(piperidin-1 yl)methanone (7bk)

Sodium hydride (60% dispersion in mineral oil, 15.7 mg, 0.39 mmol) was added to a solution of 2-fluoro-6-(trifluoromethoxy)phenol (76.8 mg, 0.39 mmol) in DMF (1 mL) at 0° C. and the mixture was stirred at 0° C. for 5-10 min. A solution of 24 (63.0 mg, 0.2 mmol) in DMF (1 mL) was then added slowly to the mixture via syringe at 0° C. The reaction mixture was stirred and allowed to warm to rt and heated to 60° C. overnight. A few drops of water were added to the mixture to quench the excess NaH and the mixture was then concentrated in vacuo. The crude residue was purified by Biotage Isolera One using 30-50% EtOAc in hexanes as eluent to give 51 mg of desired compound 7bk as a yellow solid (54% yield); Mp 159-161° C. ⁱH NMR (CDCl₃) δ 1.46 (s, 9H), 1.56-1.80 (m, 6H), 3.33-3.58 (m, 3H), 3.94 (s, 1H), 7.12-7.38 (m, 3H), 8.41 (s, 1H); HPLC (Method A): retention time 2.32 min, m/z 482.2 [M+H]⁺.

### Example 66

HEK293 cells stably transfected with human MrgprX1 were plated onto poly-D-lysine-coated, black-walled, 96-well plates at a seeding density of 12,000 cells/well and grown for two days. On the day of the experiment, cells were incubated with Fluo-4 AM 2 μM, pluronic acid 0.04% and Trypan Red 1% in HBSS (pH 7.4, 100 μL) for 1 h at 37° C. For each different passage of cells, a BAM8-22 dose response was first conducted and the BAM8-22 EC₂₀ determined. Test compounds were then dissolved and serially diluted in DMSO, further diluted with Trypan Red (1%)-containing HBSS (50 μL) and added to cells (0.33% DMSO concentration by volume) to first monitor for agonist activity. After 2.5 min, BAM8-22 at 4-fold EC₂₀ (50 μL) was added to cells (0.25% DMSO concentration by volume) and the cells imaged on the FLIPR for an additional 2 minutes to monitor the test compounds for PAM activity. Finally, data were normalized to fluorescence signals obtained from 250 nM of BAM8-22 (maximum) and BAM8-22 at its EC₂₀ concentration (minimum). EC₅₀ of a test compound was defined as the concentration required to provoke a response halfway between the minimum and maximum response in the presence of EC₂₀ concentration of BAM8-22. The results are summarized in Table 1.

TABLE 1

| | EC₅₀ |
|---|---|
| Compound^{†} | (μM) |
| 2-((5-(4-Fluorophenyl)thieno[2,3-d]pyrimidin-4-yl)oxy)-N,N- | >100 |
| dimethylacetamide (A) | |
| 6-Phenyl-4-(o-tolyloxy)furo[2,3-d]pyrimidine (B) | 1 |
| 4-(2,3-Dimethylphenoxy)-6-phenylfuro[2,3-d]pyrimidine (C) | 70 |
| 4-(o-Tolyloxy)thieno[2,3-d]pyrimidine (D) | 10 |
| 4-(p-Tolyloxy)thieno[2,3-d]pyrimidine (E) | 0.9 |
| 6-tert-Butyl-5-(3,4-dichlorophenyl)-4-(2- | 0.1 |
| (trifluoromethoxy)phenoxy)thieno[2,3-d]pyrimidine (7a) | |
| 6-tert-Butyl-5-(3,4-dichlorophenyl)-4-(2-fluoro-6- | 0.05 |
| (trifluoromethoxy)phenoxy)thieno[2,3-d]pyrimidine (7b) | |
| 6-tert-Butyl-4-(2-chloro-6-(trifluoromethoxy)phenoxy)-5-(3,4- | 0.3 |
| dichlorophenyl)thieno[2,3-d]pyrimidine (7c) | |
| 6-tert-Butyl-5-(3,4-dichlorophenyl)-4-(3- | >10 |
| (trifluoromethoxy)phenoxy)thieno[2,3-d]pyrimidine (7d) | |
| 6-tert-Butyl-5-(3,4-dichlorophenyl)-4-(4- | >100 |
| (trifluoromethoxy)phenoxy)thieno[2,3-d]pyrimidine (7e) | |
| 6-tert-Butyl-5-(3,4-dichlorophenyl)-4-(4-nitro-2- | >100 |
| (trifluoromethoxy)phenoxy)thieno[2,3-d]pyrimidine (7f) | |
| 6-tert-Butyl-5-(3,4-dichlorophenyl)-4-(2-fluoropyridin-3-yloxy)thieno[2,3- | 1 |
| d]pyrimidine (7g) | |
| 6-tert-Butyl-5-(3,4-dichlorophenyl)-4-(2-(trifluoromethoxy)pyridin-3- | 4 |
| yloxy)thieno[2,3-d]pyrimidine (7h) | |
| 6-tert-Butyl-5-(3,4-dichlorophenyl)-4-(2-methoxypyridin-3- | 10 |
| yloxy)thieno[2,3-d]pyrimidine (7i) | |
| 3-(6-tert-Butyl-5-(3,4-dichlorophenyl)thieno[2,3-d]pyrimidin-4- | 30 |
| yloxy)pyridin-2-amine (7j) | |
| 6-tert-Butyl-5-(3,4-dichlorophenyl)-4-(pyridazin-4-yloxy)thieno[2,3- | >100 |
| d]pyrimidine (7k) | |
| 6-tert-Butyl-5-(3,4-dichlorophenyl)-4-phenoxythieno[2,3-d]pyrimidine | 0.6 |
| (7l) | |
| 6-tert-Butyl-5-(3,4-dichlorophenyl)-4-(2-fluorophenoxy)thieno[2,3- | 0.1 |
| d]pyrimidine (7m) | |
| 6-tert-Butyl-5-(3,4-dichlorophenyl)-4-(2,6-difluorophenoxy)thieno[2,3- | 0.02 |
| d]pyrimidine (7n) | |
| 6-tert-Butyl-5-(3,4-dichlorophenyl)-4-(3-fluorophenoxy)thieno[2,3- | >100 |
| d]pyrimidine (7o) | |
| 6-tert-Butyl-5-(3,4-dichlorophenyl)-4-(4-fluorophenoxy)thieno[2,3- | 2 |
| d]pyrimidine (7p) | |
| 6-tert-Butyl-5-(3,4-dichlorophenyl)-4-(2-methoxyphenoxy)thieno[2,3- | 0.7 |
| d]pyrimidine (7q) | |
| 6-tert-Butyl-5-(3,4-dichlorophenyl)-4-(2-ethoxyphenoxy)thieno[2,3- | 1 |
| d]pyrimidine (7r) | |
| 6-tert-Butyl-5-(3,4-dichlorophenyl)-4-(2-(2,2,2- | 1 |
| trifluoroethoxy)phenoxy)thieno[2,3-d]pyrimidine (7s) | |
| 6-tert-Butyl-5-(3,4-dichlorophenyl)-4-(o-tolyloxy)thieno[2,3-d]pyrimidine | 0.4 |
| (7t) | |
| 1-(2-(6-tert-Butyl-5-(3,4-dichlorophenyl)thieno[2,3-d]pyrimidin-4- | 3 |
| yloxy)phenyl)ethanone (7u) | |
| 2-(6-tert-Butyl-5-(3,4-dichlorophenyl)thieno[2,3-d]pyrimidin-4- | 0.5 |
| yloxy)benzonitrile (7v) | |
| 6-tert-Butyl-5-(3,4-dichlorophenyl)-4-isopropoxythieno[2,3-d]pyrimidine | >10 |
| (7w) | |
| 6-tert-Butyl-4-cyclopropoxy-5-(3,4-dichlorophenyl)thieno[2,3- | >10 |
| d]pyrimidine (7x) | |
| 6-tert-Butyl-4-cyclobutoxy-5-(3,4-dichlorophenyl)thieno[2,3-d]pyrimidine | 7 |
| (7y) | |
| 6-tert-Butyl-4-(cyclopentyloxy)-5-(3,4-dichlorophenyl)thieno[2,3- | 6 |
| d]pyrimidine (7z) | |
| 6-tert-Butyl-4-(cyclohexyloxy)-5-(3,4-dichlorophenyl)thieno[2,3- | 0.1 |
| d]pyrimidine (7aa) | |
| 6-tert-Butyl-5-(3,4-dichlorophenyl)-4-(1-methylpiperidin-3- | 6 |
| yloxy)thieno[2,3-d]pyrimidine (7ab) | |
| 6-tert-Butyl-5-(3,4-dichlorophenyl)-4-(2-methylcyclohexyloxy)thieno[2,3- | 0.1 |
| d]pyrimidine (7ac) | |
| 4-(Bicyclo[2.2.1 ]heptan-2-yloxy)-6-tert-butyl-5-(3,4- | 0.3 |
| dichlorophenyl)thieno[2,3-d]pyrimidine (7ad) | |
| 6-tert-Butyl-4-(cycloheptyloxy)-5-(3,4-dichlorophenyl)thieno[2,3- | 0.4 |
| d]pyrimidine (7ae) | |
| 6-tert-Butyl-5-(3,4-dichlorophenyl)-4-(2- | 0.1 |
| (trifluoromethyl)cyclohexyloxy)thieno[2,3-d]pyrimidine (7af) | |
| 6-tert-Butyl-5-(3,4-dichlorophenyl)-4-(2-fluorocyclohexyloxy)thieno[2,3- | 0.1 |
| d]pyrimidine (7ag) | |
| 6-tert-Butyl-5-(3,4-dichlorophenyl)-4-(2,2- | 0.3 |
| dimethylcyclohexyloxy)thieno[2,3-d]pyrimidine (7ah) | |
| 6-tert-Butyl-5-(3,4-dichlorophenyl)-4-(1- | >10 |
| (trifluoromethyl)cyclohexyloxy)thieno[2,3-d]pyrimidine (7ai) | |
| 6-tert-Butyl-5-(3,4-difluorophenyl)-4-(2-fluorophenoxy)thieno[2,3- | 0.1 |
| d]pyrimidine (7aj) | |
| 6-tert-Butyl-4-(2,6-difluorophenoxy)-5-(3,4-difluorophenyl)thieno[2,3- | 0.07 |
| d]pyrimidine (7ak) | |
| 6-tert-Butyl-5-(3,4-difluorophenyl)-4-(2-fluoro-6- | 0.02 |
| (trifluoromethoxy)phenoxy)thieno[2,3-d]pyrimidine (7al) | |
| 6-tert-Butyl-5-(3,4-difluorophenyl)-4-(2- | 0.07 |
| (trifluoromethoxy)phenoxy)thieno[2,3-d]pyrimidine (7am) | |
| 6-tert-Butyl-5-(4-chlorophenyl)-4-(2- | 0.08 |
| (trifluoromethoxy)phenoxy)thieno[2,3-d]pyrimidine (7an) | |
| 6-tert-Butyl-5-(4-chlorophenyl)-4-(2-fluoro-6- | 0.01 |
| (trifluoromethoxy)phenoxy)thieno[2,3-d]pyrimidine (7ao) | |
| 6-tert-Butyl-5-(4-chlorophenyl)-4-(2,6-difluorophenoxy)thieno[2,3- | 0.03 |
| d]pyrimidine (7ap) | |
| 6-tert-Butyl-5-(3-chlorophenyl)-4-(2- | 0.1 |
| (trifluoromethoxy)phenoxy)thieno[2,3-d]pyrimidine (7aq) | |
| 6-tert-Butyl-5-(3-chlorophenyl)-4-(2-fluoro-6- | 0.03 |
| (trifluoromethoxy)phenoxy)thieno[2,3-d]pyrimidine (7ar) | |
| 6-tert-Butyl-5-(3-chlorophenyl)-4-(2,6-difluorophenoxy)thieno[2,3- | 0.05 |
| d]pyrimidine (7as) | |
| 6-tert-Butyl-4-(2,6-difluorophenoxy)-5-(4-fluorophenyl)thieno[2,3- | 0.07 |
| d]pyrimidine (7at) | |
| 6-tert-Butyl-4-(2-fluorophenoxy)-5-(4-fluorophenyl)thieno[2,3- | 0.3 |
| d]pyrimidine (7au) | |
| 6-tert-Butyl-5-(3-fluorophenyl)-4-(2- | 0.1 |
| (trifluoromethoxy)phenoxy)thieno[2,3-d]pyrimidine (7av) | |
| 6-tert-Butyl-4-(2,6-difluorophenoxy)-5-(3-fluorophenyl)thieno[2,3- | 0.1 |
| d]pyrimidine (7aw) | |
| 6-tert-Butyl-5-(3,4-dichlorophenyl)-4-(2- | 0.2 |
| (trifluoromethoxy)phenylthio)thieno[2,3-d]pyrimidine (7ax) | |
| 2-methyl-4-phenoxythieno[2,3-d]pyrimidine (7ay) | 6 |
| 5,6-Dimethyl-4-(o-tolyloxy)thieno[2,3-d]pyrimidine (7az) | 0.5 |
| 4-(2-Fluorophenoxy)-5,6-dimethylthieno[2,3-d]pyrimidine (7ba) | 0.2 |
| 4-(2-Fluorophenoxy)-6-methyl-5-phenylthieno[2,3-d]pyrimidine (7bb) | 0.4 |
| 6-(tert-Butyl)-4-(2-fluorophenoxy)thieno[2,3-d]pyrimidine (7bc) | 0.4 |
| 6-(tert-Butyl)-4-(2-fluorophenoxy)-5-phenylthieno[2,3-d]pyrimidine (7bd) | 0.3 |
| Ethyl 6-(tert-buty1)-4-(2-fluoro-6-(trifluoromethoxy)phenoxy)thieno[2,3- | 0.06 |
| d]pyrimidine-5-carboxylate (7be) | |
| 6-(tert-Butyl)-4-(2-fluoro-6-(trifluoromethoxy)phenoxy)-5-(pyrrolidin-1- | 0.03 |
| yl)thieno[2,3-d]pyrimidine (7bf) | |
| 6-(tert-Butyl)-4-(2-fluoro-6-(trifluoromethoxy)phenoxy)-5-(2- | 0.07 |
| methylpyrrolidin-1-yl)thieno[2,3-d]pyrimidine (7bg) | |
| 6-(tert-Butyl)-4-(2-fluoro-6-(trifluoromethoxy)phenoxy)-5-(piperidin-1- | 0.04 |
| yl)thieno[2,3-d]pyrimidine (7bh) | |
| 4-(6-(tert-Butyl)-4-(2,6-difluorophenoxy)thieno[2,3-d]pyrimidin-5- | 0.04 |
| yl)morpholine (7bi) | |
| 6-(tert-Butyl)-N,N-diethyl-4-(2-fluoro-6- | 0.07 |
| (trifluoromethoxy)phenoxy)thieno[2,3-d]pyrimidin-5-amine (7bj) | |
| (6-(tert-Butyl)-4-(2-fluoro-6-(trifluoromethoxy)phenoxy)furo[2,3- | 0.1 |
| d]pyrimidin-5-yl)(piperidin-1-yl)methanone (7bk) | |

^{†}Compounds (A) (PubChem CID: 2088075), (B) (PubChem CID: 3825639), (C) (PubChem CID: 3775786), (D) (PubChem CID: 710974), (E) (PubChem CID: 710977), and (7ay) (PubChem CID: 1233965) were previosouly reported by the National Center for Biotechnology Information. “PubChem Bioassay Record for AID 602413, Source: Johns Hopkins Ion Channel Center” PubChem, pubchem.ncbi.nlm.nih.gov/bioassay/602413. Accessed 12 Nov. 2020.

### Example 67

### In Vitro Metabolic Stability Studies

The metabolic stability was evaluated using mouse liver microsomes. For the cytochrome P450 (CYP)-mediated metabolism, the reaction was carried out with 100 mM potassium phosphate buffer, pH 7.4, in the presence of NADPH regenerating system (1.3 mM NADPH, 3.3 mM glucose 6-phosphate, 3.3 mM MgCl₂, 0.4 U/mL glucose-6-phosphate dehydrogenase, 50 μM sodium citrate). Reactions, in triplicate, were initiated by addition of the liver microsomes to the incubation mixture (compound final concentration was 5 μM; 0.5 mg/mL microsomes). After 60 min of incubation, aliquots of the mixture were removed and the reaction quenched by addition of three times the volume of ice-cold acetonitrile spiked with the internal standard. Compound disappearance was monitored over time using a liquid chromatography and tandem mass spectrometry (LC/MS/MS) method. The results are summarized in Table 2.

TABLE 2
Metabolic stability in liver microsomes

| | % remaining after | |
|---|---|---|
| | 1-h incubation | |
| | Mouse | Human |
| 6-tert-Butyl-5-(3,4-dichlorophenyl)-4-(2- | 25 | >95 |
| (trifluoromethoxy)phenoxy)thieno[2,3-d]pyrimidine (7a) | | |
| 5,6-Dimethyl-4-(o-tolyloxy)thieno[2,3-d]pyrimidine (7az) | <5 | ND |
| 4-(2-Fluorophenoxy)-5,6-dimethylthieno[2,3-d]pyrimidine (7ba) | <5 | <5 |
| 4-(2-Fluorophenoxy)-6-methyl-5-phenylthieno[2,3-d]pyrimidine | <5 | 12 |
| (7bb) | | |
| 6-(tert-Butyl)-4-(2-fluorophenoxy)thieno[2,3-d]pyrimidine (7bc) | <5 | 61 |
| 6-(tert-Butyl)-4-(2-fluorophenoxy)-5-phenylthieno[2,3- | <5 | 78 |
| d]pyrimidine (7bd) | | |
| 6-tert-Butyl-5-(3,4-dichlorophenyl)-4-(2,6- | 36 | 93 |
| difluorophenoxy)thieno[2,3-d]pyrimidine (7n) | | |
| 6-tert-Butyl-5-(3,4-dichlorophenyl)-4-(2-fluoro-6- | 74 | >95 |
| (trifluoromethoxy)phenoxy)thieno[2,3-d]pyrimidine (7b) | | |
| 6-tert-Butyl-5-(3,4-difluorophenyl)-4-(2-fluoro-6- | >95 | >95 |
| (trifluoromethoxy)phenoxy)thieno[2,3-d]pyrimidine (7al) | | |
| 6-tert-Butyl-5-(4-chlorophenyl)-4-(2-fluoro-6- | >95 | >95 |
| (trifluoromethoxy)phenoxy)thieno[2,3-d]pyrimidine (7ao) | | |

### Example 68

### In Vivo Pharmacokinetics

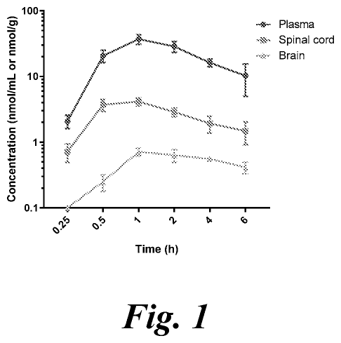

Male CD-1 mice between 25 and 30 g were obtained from Harlan, and maintained on a 12-h light-dark cycle with ad libitum access to food and water. Three animals were used per time-point for each treatment group. Compound 7a was dissolved in 5% dimethyl acetamide, 10% Tween80, 10% Cremophor EL, 25% PEG400, and 50% water, and administered to male mice as a single oral dose by oral gavage of 100 mg/kg. The mice were sacrificed at various time points post-drug administration. Animals were euthanized with CO₂, and blood samples were collected in heparinized microtubes by cardiac puncture. Brain and spinal cord tissues were dissected and immediately flash frozen (−80° C.). Blood samples were spun at 2,000×g for 15 min, plasma was removed and stored at −80° C. until LC-MS analysis. Prior to extraction, frozen samples were thawed on ice. The calibration curves were developed using plasma and brain from naïve animals as a matrix. Plasma samples (50 μL), were processed using a single liquid extraction method by addition of 300 μL of acetonitrile as with internal standard (losartan: 0.5 μM), followed by vortex mixing for 30 sec and then centrifugation at 10,000×g for 10 min at 4° C. Fifty microliter of the supernatant is diluted with 50 μL of water and transferred to 250 μL polypropylene autosampler vials sealed with a Teflon cap. For brain and spinal cord tissue analysis, samples were weighed in the range of (80-100 mg) to which 2-volumes of acetonitrile as with internal standard (losartan: 0.5 μM) was added and homogenized for extraction of the analyte. Samples were vortex mixed for 1 min and centrifuged as above. A 20 μL aliquot of supernatant was diluted with 20 μL of water and transferred to 250 μL polypropylene autosampler vials sealed with a Teflon cap. A volume of 3 μL was injected onto the ultra-performance liquid chromatography (UPLC) instrument for quantitative analysis by LC-MS/MS. Samples were analyzed on a Thermo Scientific Accela UPLC system coupled to Accela open autosampler at ambient temperature with an Agilent Eclipse Plus column (100×2.1 mm i.d.) packed with a 1.8 μm C18 stationary phase. The autosampler was temperature controlled and operated at 10° C. The mobile phase used for the chromatographic separation was composed of acetonitrile/water containing 0.1% formic acid and with a flow rate of 0.4 mL/min for 4.5 min using gradient elution. The column effluent was monitored using TSQ Vantage triple-quadrupole mass-spectrometric detector, equipped with an electrospray probe set in the positive ionization mode. Samples were introduced into the ionization source through a heated nebulized probe (350° C.). Drug concentration versus time curves are shown in FIG. 1. Pharmacokinetics parameters are summarized in Table 3.

TABLE 3
Oral pharmacokinetics parameters of
compound 7a (100 mg/kg) in mice

| | Tₘₐₓ | Cₘₐₓ | AUC₀₋ₗₐₛₜ |
|---|---|---|---|
| Matrix | (h) | (nmol/mL) | (nmol*h/mL) |
| Plasma | 1.67 ± 0.33 | 38.98 ± 5.06 | 108.53 ± 1.18 |
| Brain | 1.67 ± 0.33 | 0.82 ± 0.05 | 2.37 ± 0.03 |
| Spinal cord | 1.17 ± 0.44 | 4.60 ± 0.60 | 13.79 ± 0.14 |

### Example 69

### Chronic Constrictive Injury (CCI) Model

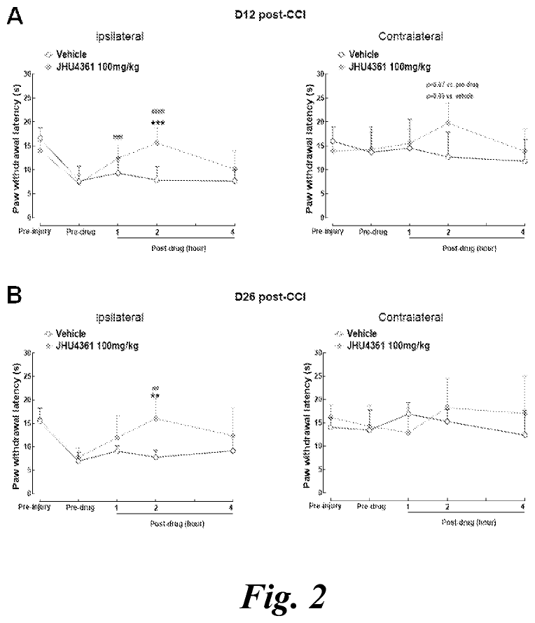

Compound 7a was examined for its ability to attenuate heat hypersensitivity in MrgprX1 mice after receiving sciatic CCI, according to the previously described methods (Proc Natl Acad Sci USA. 2017 Mar. 7; 114(10):E1996-E2005). Paw withdrawal latency (PWL) to noxious heat stimuli was measured with the Hargreaves test. The results are summarized in FIG. 2 (** P<0.01, *** P<0.001 vs. vehicle; ##P<0.01, ###P<0.001, ####P<0.0001 vs. pre-drug. Two-way mixed-model ANOVA with Bonferroni post hoc test. Data are expressed as mean+SD). PWLs to noxious heat stimuli in the ipsilateral (left) hind paw to the side of nerve injury were decreased at day 12 (FIG. 2A) and day 26 post-CCI (FIG. 2B), suggesting the development of heat hypersensitivity. PWLs in contralateral (right panel) hind paw were not significantly decreased from pre-injury baselines at day 12 (FIG. 2A) and day 26 (FIG. 2B) after CCI. The ipsilateral PWLs were significantly increased from pre-drug level at 1-2 hours after receiving compound 7a (100 mg/kg, N=5,3 males, 2 females), but not vehicle (5% Dimethyl acetamide+10% Cremophor EL+10% Tween80+25% PEG400+50% water, 10 mL/kg, N=4, 2 males, 2 females), through oral gavage. There were no significant changes in contralateral PWL after compound 7a treatment, as compared to pre-drug baseline or vehicle-treated group.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Dong X, Han S, Zylka MJ, Simon MI, Anderson DJ. A diverse family of GPCRs expressed in specific subsets of nociceptive sensory neurons. Cell. 2001; 106(5):619-32.
Lembo PM, Grazzini E, Groblewski T, O'Donnell D, Roy MO, Zhang J, Hoffert C, Cao J, Schmidt R, Pelletier M, Labarre M, Gosselin M, Fortin Y, Banville D, Shen SH, Strom P, Payza K, Dray A, Walker P, Ahmad S. Proenkephalin A gene products activate a new family of sensory neuron--specific GPCRs. Nat Neurosci. 2002; 5(3):201-9.
Zhang L, Taylor N, Xie Y, Ford R, Johnson J, Paulsen JE, Bates B. Cloning and expression of MRG receptors in macaque, mouse, and human. Brain Res Mol Brain Res. 2005; 133(2):187-97.
Wen W, Wang Y, Li Z, Tseng PY, McManus O B, Wu M, Li M, Lindsley CW, Dong X, Hopkins CR. Discovery and characterization of 2-(cyclopropanesulfonamido)-N-(2-ethoxyphenyl)benzamide, ML382: a potent and selective positive allosteric modulator of MrgX1. ChemMedChem. 2015; 10(1):57-61.
Li Z, Tseng PY, Tiwari V, Xu Q, He SQ, Wang Y, Zheng Q, Han L, Wu Z, Blobaum AL, Cui Y, Tiwari V, Sun S, Cheng Y, Huang-Lionnet JH, Geng Y, Xiao B, Peng J, Hopkins C, Raja SN, Guan Y, Dong X. Targeting human Mas-related G protein-coupled receptor X1 to inhibit persistent pain. Proc Natl Acad Sci USA. 2017;114(10):E1996-E2005.
National Center for Biotechnology Information. “PubChem Bioassay Record for AID 602413, Source: Johns Hopkins Ion Channel Center” PubChem, pubchem.ncbi.nlm.nih.gov/bioassay/602413. Accessed 12 Nov. 2020.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 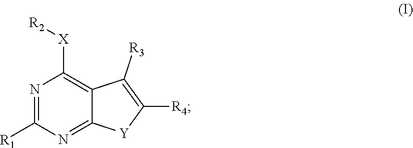
wherein:
X and Y are each independently O or S;
R₁ is selected from the group consisting of H, C₁-C₄ alkyl, C₃-C₇ substituted or unsubstituted cycloalkyl or cycloheteroalkyl, substituted or unsubstituted aryl, and substituted or unsubstituted heteroaryl;
R₂ is selected from the group consisting of C₁-C₄ alkyl, C₃-C₇ substituted or unsubstituted cycloalkyl or cycloheteroalkyl, substituted or unsubstituted aryl, substituted or unsubstituted arylalkyl, and substituted or unsubstituted heteroaryl;
R₃ is selected from the group consisting of H, C₁-C₄ alkyl, C₃-C₇ substituted or unsubstituted cycloalkyl or cycloheteroalkyl, substituted or unsubstituted aryl, substituted or unsubstituted heteroaryl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloalkyl or cycloheteroalkyl, substituted or unsubstituted aryl, substituted or unsubstituted arylalkyl, substituted or unsubstituted heteroaryl, and —OR₈, wherein R₈ is C₁-C₄ alkyl;
R₄ is selected from the group consisting of straightchain or branched substituted or unsubstituted C₁-C₄ alkyl, C₃-C₇ substituted or unsubstituted cycloalkyl or cycloheteroalkyl, substituted or unsubstituted aryl, and substituted or unsubstituted heteroaryl;
provided that R₂ and R₃ or R₃ and R₄, or any substituent groups thereof, do not together form a cyclic ring;
under the further provisos:
(a) when X is O and Y is S:
(i) R₄ is not a methyl or ethyl group;
(ii) when R₃ is H and R₄ is a t-butyl group, R₂ cannot be (3-substituted isozazol-5-yl)methyl;
(iii) when R₄ is an isopropyl group, R₂ cannot be a substituted cyclohexyl group; and
(iv) when R₄ is a butyl group or a 2-oxopropyl group, R₃ cannot be H;
(b) when X and Y are both S:
(i) when R₄ is an isopropyl group, R₂ cannot be carboxymethyl or 2-methoxy-2-oxoethyl;
(ii) when R₄ is a cyclopropyl group, R₂ cannot be carboxymethyl or 2-methoxy-2-oxoethyl;
(iii) when R₄ is a propyl group, R₃ cannot be chloride; and
(iv) when R₄ is a 3-ethoxy-3-oxopropyl group, R₃ cannot be H;
(c) when X and Y are both O:
(i) when R₄ cannot be a methyl group; and
(ii) when R₄ is a cyclopentyl group, R₃ cannot be 4-methoxyphenyl; and
(d) when X is S and Y is O:
(i) when R₃ is H, R₂ cannot be methyl; and
(ii) when R₃ is methyl, R₂ cannot be carboxymethyl, 2-methoxy-2-oxoethyl, 4-fluorophenyl, or 2-(3-methyl-4-oxoimidazolidin-1-yl)-2-oxoethyl; and
pharmaceutically acceptable salts thereof.

2. The compound of claim 1, wherein R₂ is selected from the group consisting of benzyl, phenyl, pyridyl, pyridazinyl, pyrimidinyl, pyrazinyl, pyrrolidinyl, piperidinyl, piperazinyl, and morpholinyl, each of which can be substituted or unsubstituted and wherein the piperazinyl is optionally substituted in the 4-nitrogen position with C₁-C₄ alkyl or acyl.

3. The compound of claim 2, wherein the benzyl, phenyl, pyridyl, pyridazinyl, pyrimidinyl, pyrazinyl, pyrrolidinyl, piperidinyl, piperazinyl, and morpholinyl are substituted with one or more of halogen, —CF₃, and —OCF₃.

4. The compound of claim 1, wherein R₃ is selected from the group consisting of phenyl, pyridyl, pyridazinyl, pyrimidinyl, pyrazinyl, oxazolyl, thiazolyl, pyrrolidinyl, piperidinyl, piperazinyl, and morpholinyl, each of which can be substituted or unsubstituted and wherein the piperazinyl is optionally substituted in the 4-nitrogen position with C₁-C₄ alkyl or acyl.

5. The compound of claim 4, wherein the phenyl, pyridyl, pyridazinyl, pyrimidinyl, pyrazinyl, oxazolyl, thiazolyl, pyrrolidinyl, piperidinyl, piperazinyl, and morpholinyl are substituted with one or more of C₁-C₄, halogen, —CF₃, and —OCF₃.

6. The compound of claim 1, wherein R₃ is selected from the group consisting of C₃-C₇ substituted or unsubstituted cycloheteroalkyl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloheteroalkyl, and —OR₈, wherein R₈ is C₁-C₄ alkyl.

7. The compound of claim 1, X is O, R₄ is t-butyl, and the compound of formula (I) is: 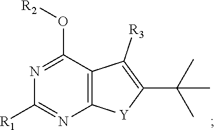
wherein:
Y is O or S;
R₁ is selected from the group consisting of H, C₁-C₄ alkyl, amino, and substituted or unsubstituted heteroaryl;
R₂ is selected from the group consisting of C₁-C₄ alkyl, C₃-C₇ substituted or unsubstituted cycloalkyl, and substituted or unsubstituted aryl;
R₃ is selected from the group consisting of H, C₁-C₄ alkyl, C₃-C₇ substituted or unsubstituted cycloalkyl or cycloheteroalkyl, substituted or unsubstituted aryl, substituted or unsubstituted heteroaryl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloalkyl or cycloheteroalkyl, substituted or unsubstituted aryl, substituted or unsubstituted arylalkyl, substituted or unsubstituted heteroaryl, and —OR₈, wherein R₈ is C₁-C₄ alkyl;
under the proviso that R₁ and R₃ cannot both be H if R₂ is methyl or 3-substituted (isoxazole-5-yl)methyl; and
pharmaceutically acceptable salt thereof.

8. The compound of claim 7, wherein the compound of formula (I) is: 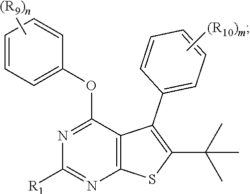
wherein:
m and n are each independently an integer selected from the group consisting of 0, 1, 2, 3, 4, and 5;
R₁ is selected from the group consisting of H, C₁-C₄ alkyl, amino, and substituted or unsubstituted heteroaryl;
each R₉ is independently selected from the group consisting of H, C₁-C₄ alkyl, halogen, —CF₃, —OCF₃, C₁-C₄ alkoxyl, substituted or unsubstituted aryl, substituted or unsubstituted cycloheteroalkyl, substituted or unsubstituted aryloxyl, and —C(═O)—R₁₁, wherein R₁₁ is C₁-C₄ alkyl; and
each R₁₀ is independently selected from the group consisting of halogen, C₁-C₄ alkyl, —CF₃, —OCF₃, C₁-C₄ alkoxyl, and one or more electron withdrawing groups selected from the group consisting of trifluoromethylsulfonyl, nitro, sulfonic acid, SO₂R₁₂, cyano, formyl, —C(═O)—R₁₃, carboxyl, —CO₂R₁₄, aminocarbonyl, and nitroso, wherein R₁₂, R₁₃ and R₁₄ are each independently C₁-C₄ alkyl.

9. The compound of claim 7, wherein R₃ is phenyl substituted with one or more halogens.

10. The compound of claim 9, wherein R₃ is selected from the group consisting of 3-chlorophenyl, 4-chlorophenyl, 3-fluorophenyl, 4-fluorophenyl, 3,4-dichlorophenyl, and 3,4-difluorophenyl.

11. The compound of claim 8, wherein R₂ is selected from the group consisting of phenyl, pyridinyl, and pyridazinyl, wherein the phenyl, pyridinyl, and pyridazinyl can be unsubstituted or substituted with one or more substituents selected from the group consisting of C₁-C₄ branched or straightchain alkyl, halogen, trifluoromethoxyl, 2,2,2-trifluoroethoxyl, nitro, C₁-C₄-alkoxyl, amino, cyano, substituted or unsubstituted C₃-C₇ cycloalkyl or cycloheteroalkyl, bicycloalkyl, and —C(═O)—R₄, wherein R₄ is C₁-C₄ alkyl.

12. The compound of claim 11, wherein R₂ is selected from the group consisting of isopropyl, 2-methylphenyl, phenyl, 2-fluorophenyl, 3-fluorophenyl, 4-fluorophenyl, 2,6-difluorophenyl, 2-fluoro-6-trifluoromethoxyphenyl, 2-trifluoromethoxy-6-fluorophenyl, 2-methoxyphenyl, 2-ethoxyphenyl, 2-trifluoromethoxyphenyl, 2-(2,2,2-trifluoroethoxy)phenyl, 2-phenylethan-1-one, 2-cyanophenyl, 3-trifluoromethoxyphenyl, 4-trifluoromethoxyphenyl, 2-fluoro-6-trifluoromethoxyphenyl, 2-chloro-6-trifluoromethoxyphenyl, 4-nitro-2-trifluoromethoxyphenyl, 4-(4-(piperazin-1-yl)phenoxy)phenyl, 4-(4-(trifluoromethyl)phenoxy)phenyl, 2-fluoropyridin-3-yl, 2-(trifluoromethoxy)pyridin-3-yl, 2-methoxypyridin-3-yl, 2-aminopyridin-3-yl, pyridazin-4-yl, cyclopropyl, cyclobutyl, cyclopentyl, cyclohexyl, cycloheptyl, 1-methylpiperidin-3-yl, 2-methylcyclohexyl, 2-trifluoromethylcyclohexyl, 2-fluorocyclohexyl, 2,2-dimethylcyclohexyl, and 1-trifluoromethylcyclohexyl, bicyclo[2.2.1]heptan-2-yl.

13. The compound of claim 7, wherein the compound of formula (I) is: 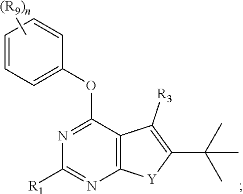
wherein:
n is 2;
Y is O or S;
R₁ is H;
each R₉ is independently F or —OF₃; and
R₃ is selected from the group consisting of C₃-C₇ substituted or unsubstituted cycloheteroalkyl, —NR₅R₆, wherein R₅ and R₆ are each independently H or C₁-C₄ alkyl, and —C(═O)—R₇, wherein R₇ is selected from C₁-C₄ alkyl, C₃-C₇ substituted or unsubstituted cycloheteroalkyl, and —OR₈, wherein R₈ is C₁-C₄ alkyl.

14. The compound of claim 1, wherein the compound is selected from the group consisting of:
6-(tert-butyl)-4-(2-fluorophenoxy)thieno[2,3-d]pyrimidine;
6-(tert-butyl)-4-(2-fluorophenoxy)-5-phenylthieno[2,3-d]pyrimidine;
6-tert-butyl-5-(3,4-dichlorophenyl)-4-(2-(trifluoromethoxy)phenoxy)thieno[2,3-d]pyrimidine;
6-tert-butyl-5-(3,4-dichlorophenyl)-4-(2-fluoro-6-(trifluoromethoxy)phenoxy)thieno[2,3-d]pyrimidine;
6-tert-butyl-4-(2-chloro-6-(trifluoromethoxy)phenoxy)-5-(3,4-dichlorophenyl)thieno[2,3-d]pyrimidine;
6-tert-butyl-5-(3,4-dichlorophenyl)-4-(3-(trifluoromethoxy)phenoxy)thieno[2,3-d]pyrimidine;
6-tert-butyl-5-(3,4-dichlorophenyl)-4-(4-(trifluoromethoxy)phenoxy)thieno[2,3-d]pyrimidine;
6-tert-butyl-5-(3,4-dichlorophenyl)-4-(4-nitro-2-(trifluoromethoxy)phenoxy)thieno[2,3-d]pyrimidine;
6-tert-butyl-5-(3,4-dichlorophenyl)-4-(2-fluoropyridin-3-yloxy)thieno[2,3-d]pyrimidine;
6-tert-butyl-5-(3,4-dichlorophenyl)-4-(2-(trifluoromethoxy)pyridin-3-yloxy)thieno[2,3-d]pyrimidine;
6-tert-butyl-5-(3,4-dichlorophenyl)-4-(2-methoxypyridin-3-yloxy)thieno[2,3-d]pyrimidine;
3-(6-tert-butyl-5-(3,4-dichlorophenyl)thieno[2,3-d]pyrimidin-4-yloxy)pyridin-2-amine;
6-tert-butyl-5-(3,4-dichlorophenyl)-4-(pyridazin-4-yloxy)thieno[2,3-d]pyrimidine;
6-tert-butyl-5-(3,4-dichlorophenyl)-4-phenoxythieno[2,3-d]pyrimidine;
6-tert-butyl-5-(3,4-dichlorophenyl)-4-(2-fluorophenoxy)thieno[2,3-d]pyrimidine;
6-tert-butyl-5-(3,4-dichlorophenyl)-4-(2,6-difluorophenoxy)thieno[2,3-d]pyrimidine;
6-tert-butyl-5-(3,4-dichlorophenyl)-4-(3-fluorophenoxy)thieno[2,3-d]pyrimidine;
6-tert-butyl-5-(3,4-dichlorophenyl)-4-(4-fluorophenoxy)thieno[2,3-d]pyrimidine;
6-tert-butyl-5-(3,4-dichlorophenyl)-4-(2-methoxyphenoxy)thieno[2,3-d]pyrimidine;
6-tert-butyl-5-(3,4-dichlorophenyl)-4-(2-ethoxyphenoxy)thieno[2,3-d]pyrimidine;
6-tert-butyl-5-(3,4-dichlorophenyl)-4-(2-(2,2,2-trifluoroethoxy)phenoxy)thieno[2,3-d]pyrimidine;
6-tert-butyl-5-(3,4-dichlorophenyl)-4-(o-tolyloxy)thieno[2,3-d]pyrimidine;
1-(2-(6-tert-butyl-5-(3,4-dichlorophenyl)thieno[2,3-d]pyrimidin-4-yloxy)phenyl)ethanone;
2-(6-tert-butyl-5-(3,4-dichlorophenyl)thieno[2,3-d]pyrimidin-4-yloxy)benzonitrile;
6-tert-butyl-5-(3,4-dichlorophenyl)-4-isopropoxythieno[2,3-d]pyrimidine;
6-tert-butyl-4-cyclopropoxy-5-(3,4-dichlorophenyl)thieno[2,3-d]pyrimidine;
6-tert-butyl-4-cyclobutoxy-5-(3,4-dichlorophenyl)thieno[2,3-d]pyrimidine;
6-tert-butyl-4-(cyclopentyloxy)-5-(3,4-dichlorophenyl)thieno[2,3-d]pyrimidine;
6-tert-butyl-4-(cyclohexyloxy)-5-(3,4-dichlorophenyl)thieno[2,3-d]pyrimidine;
6-tert-butyl-5-(3,4-dichlorophenyl)-4-(1-methylpiperidin-3-yloxy)thieno[2,3-d]pyrimidine;
6-tert-butyl-5-(3,4-dichlorophenyl)-4-(2-methylcyclohexyloxy)thieno[2,3-d]pyrimidine;
4-(Bicyclo[2.2.1]heptan-2-yloxy)-6-tert-butyl-5-(3,4-dichlorophenyl)thieno[2,3-d]pyrimidine;
6-tert-butyl-4-(cyclohepyloxy)-5-(3,4-dichlorophenyl)thieno[2,3-d]pyrimidine;
6-tert-butyl-5-(3,4-dichlorophenyl)-4-(2-(trifluoromethyl)cyclohexyloxy)thieno[2,3-d]pyrimidine;
6-tert-butyl-5-(3,4-dichlorophenyl)-4-(2-fluorocyclohexyloxy)thieno[2,3-d]pyrimidine;
6-tert-butyl-5-(3,4-dichlorophenyl)-4-(2,2-dimethylcyclohexyloxy)thieno[2,3-d]pyrimidine;
6-tert-butyl-5-(3,4-dichlorophenyl)-4-(1-(trifluoromethyl)cyclohexyloxy)thieno[2,3-d]pyrimidine;
6-tert-butyl-5-(3,4-difluorophenyl)-4-(2-fluorophenoxy)thieno[2,3-d]pyrimidine;
6-tert-butyl-4-(2,6-difluorophenoxy)-5-(3,4-difluorophenyl)thieno[2,3-d]pyrimidine;
6-tert-butyl-5-(3,4-difluorophenyl)-4-(2-fluoro-6-(trifluoromethoxy)phenoxy)thieno[2,3-d]pyrimidine;
6-tert-butyl-5-(3,4-difluorophenyl)-4-(2-(trifluoromethoxy)phenoxy)thieno[2,3-d]pyrimidine;
6-tert-butyl-5-(4-chlorophenyl)-4-(2-(trifluoromethoxy)phenoxy)thieno[2,3-d]pyrimidine;
6-tert-butyl-5-(4-chlorophenyl)-4-(2-fluoro-6-(trifluoromethoxy)phenoxy)thieno[2,3-d]pyrimidine;
6-tert-butyl-5-(4-chlorophenyl)-4-(2,6-difluorophenoxy)thieno[2,3-d]pyrimidine;
6-tert-butyl-5-(3-chlorophenyl)-4-(2-(trifluoromethoxy)phenoxy)thieno[2,3-d]pyrimidine;
6-tert-butyl-5-(3-chlorophenyl)-4-(2-fluoro-6-(trifluoromethoxy)phenoxy)thieno[2,3-d]pyrimidine;
6-tert-butyl-5-(3-chlorophenyl)-4-(2,6-difluorophenoxy)thieno[2,3-d]pyrimidine;
6-tert-butyl-4-(2,6-difluorophenoxy)-5-(4-fluorophenyl)thieno[2,3-d]pyrimidine;
6-tert-butyl-4-(2-fluorophenoxy)-5-(4-fluorophenyl)thieno[2,3-d]pyrimidine;
6-tert-butyl-5-(3-fluorophenyl)-4-(2-(trifluoromethoxy)phenoxy)thieno[2,3-d]pyrimidine;
6-tert-butyl-4-(2,6-difluorophenoxy)-5-(3-fluorophenyl)thieno[2,3-d]pyrimidine;
6-tert-butyl-5-(3,4-dichlorophenyl)-4-(2-(trifluoromethoxy)phenylthio)thieno[2,3-d]pyrimidine;
ethyl 6-(tert-butyl)-4-(2-fluoro-6-(trifluoromethoxy)phenoxy)thieno[2,3-d]pyrimidine-5-carboxylate;
6-(tert-butyl)-4-(2-fluoro-6-(trifluoromethoxy)phenoxy)-5-(pyrrolidin-1-yl)thieno[2,3-d]pyrimidine;
6-(tert-butyl)-4-(2-fluoro-6-(trifluoromethoxy)phenoxy)-5-(2-methylpyrrolidin-1-yl)thieno[2,3-d]pyrimidine;
6-(tert-butyl)-4-(2-fluoro-6-(trifluoromethoxy)phenoxy)-5-(piperidin-1-yl)thieno[2,3-d]pyrimidine;
4-(6-(tert-butyl)-4-(2,6-difluorophenoxy)thieno[2,3-d]pyrimidin-5-yl)morpholine;
6-(tert-butyl)-N,N-diethyl-4-(2-fluoro-6-(trifluoromethoxy)phenoxy)thieno[2,3-d]pyrimidin-5-amine; and
(6-(tert-butyl)-4-(2-fluoro-6-(trifluoromethoxy)phenoxy)furo[2,3-d]pyrimidin-5-yl)(piperidin-1-yl)methanone.

15. The compound of claim 1, wherein X is S, Y is S, and R₄ is t-butyl and the compound of formula (I) is:

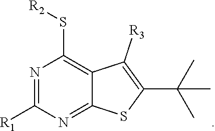

16. The compound of claim 15, wherein the compound of formula (I) is:

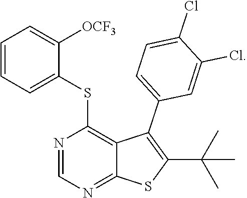 [SMILES: CC(C)(C)c1sc2ncnc(Sc3ccccc3OC(F)(F)F)c2c1-c1ccc(Cl)c(Cl)c1]

17. The compound of claim 1, wherein X is O and Y is O and the compound of formula (I) is:

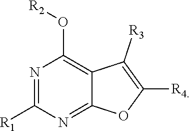

18. The compound of claim 1, wherein X is S and Y is O and the compound of formula (I) is:

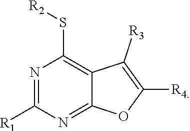

19. A method for treating pain in a subject in need of treatment thereof, the method comprising administering to the subject a compound of any one of claims 1-18, or a pharmaceutically acceptable salt thereof, in a therapeutically effective amount to treat the pain.

20. The method of claim 19, wherein the pain comprises neuropathic pain.

21. The method of claim 20, wherein the neuropathic pain comprises chronic neuropathic pain.

22. The method of claim 20, wherein the neuropathic pain is selected from the group consisting of chemotherapy-induced pain, post-traumatic injury pain, crush pain, painful traumatic mononeuropathy, painful polyneuropathy, pain resulting from spinal injury, nerve compression or entrapment, sacral pain, trigeminal neuralgia, migraine and migraine headache, postherpetic neuralgia, phantom limb pain, diabetic neuropathy, including diabetic peripheral neuropathic pain, postamputation pain, lumbar radiculopathy, and complex regional pain syndromes.

23. The method of claim 19, further comprising alleviating or attenuating pain.

24. The method of claim 19, further comprising eliminating or attenuating one or more of off-target side effects, opioid-like side effects, and abuse potential.

25. The method of claim 24, wherein the off-target side effect comprises itching.

26. The method of claim 19, wherein the compound comprises a positive allosteric modulator of MRGPRX1.

27. The method of claim 26, wherein the MRGPRX1 is expressed in one or more DRG neurons.

28. The method of claim 19, further comprising administering one or more additional therapeutic agents in combination with a compound of any one of claims 1-16.

29. The method of claim 28, wherein the one or more additional therapeutic agents are selected from the group consisting of a therapeutic agent for pain and an anti-inflammatory agent.

30. The method of claim 29, wherein the therapeutic agent for pain comprises an opioid agent selected from the group consisting of morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene.

31. The method of claim 29, wherein the therapeutic agent for pain is a non-opioid analgesic agents selected from the group consisting of aspirin, acetaminophen, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

32. The method of claim 28, wherein the one or more therapeutic agents are selected from the group consisting of a neuropathic disorder agent, an antidepressant, a regional anesthetic, ketamine, and combinations thereof.

33. The method of claim 28, wherein the one or more therapeutic agents comprise an anti-inflammatory agent selected from the group consisting of a steroid, an antihistamine, and combinations thereof.

34. The method of claim 19, wherein the compound is administered systemically.

35. The method of claim 34, wherein the systemic administration is selected from the group consisting of oral, buccal, sublingual, nasal, via an inhaler, suppository, topical, transdermal, intradermal, subcutaneous, intramuscular, intravenous, and intraperitoneal.

36. The method of any of claims 19-35, wherein the compound penetrates the central nervous system.
